(19) United States
(12) Patent Application Publication
(10) Pub. No.: US 2018/0310854 A1
(43) Pub. Date: Nov. 1, 2018

(54) METHOD AND SYSTEM FOR ESTIMATING POTENTIAL DISTRIBUTION ON CORTICAL SURFACE

(71) Applicants: Elminda Ltd., Herzliya (IL); B.G. Negev Technologies & Applications Ltd., at Ben-Gurion University, Beer-Sheva (IL)

(72) Inventors: Amir B. GEVA, Tel-Aviv (IL); Reuven SHAVIT, Pardesia (IL); Drod HAOR, Kibbutz Hulda (IL); Yaki STERN, Moshav Sde Yaakov (IL); Ziv PEREMEN, Natanya (IL)

(21) Appl. No.: 15/772,151

(22) PCT Filed: Nov. 1, 2016
(86) PCT No.: PCT/IL2016/051181

§ 371 Date: Apr. 30, 2018

Related U.S. Application Data
(60) Provisional application No. 62/249,293, filed on Nov. 1, 2015.

(51) Int. Cl. A61B 5/0478; A61B 5/04; A61B 5/00

(52) U.S. Cl. CPC A61B 5/0478; A61B 5/04014; A61B 5/7242; A61B 5/7264; A61B 5/1077; A61B 5/7246; A61B 5/6814; A61B 5/7207; A61B 5/7239

(57) ABSTRACT

A method of estimating potential distribution over a cortical surface of a brain of a subject is disclosed. The method comprises: obtaining encephalogram (EG) data recorded from a scalp surface of the head, and head model data describing a geometry of the head and electrical property distribution of tissues within the head. The method further comprises calculating differentials of the EG data over the scalp surface, calculating volumetric distribution of electrical potential between the cortex and scalp surfaces using the EG data and the differentials, and estimating the potential distribution over the cortical surface based on the volumetric distribution.

### FIELD AND BACKGROUND OF THE INVENTION

The present invention, in some embodiments thereof, relates to medical imaging and, more particularly, but not exclusively, to a method and system for generating an image describing an estimate of the potential distribution on the cortical surface.

Electroencephalogram (EEG) based techniques are widely used for non-invasive monitoring of electrical brain activity by measuring electrical signals on the scalp. The advantage of this method is a direct spatial measurement of the potentials, high temporal resolution and simplicity of the sensor.

Known in the art are methods which seek to estimate the potential distribution on the cortical surface using only scalp potentials measured by EEG. One such method is a current estimation method which employs a two-dimensional image sharpening procedure utilizing the Surface Laplacian (SL) [Nunez and Srinivasan, Electric Fields of the Brain: The Neurophysics of EEG, 2nd Ed. New York: Oxford University Press, 2006; Perrin et al., “Scalp current density mapping: value and estimation from potential data”, IEEE Trans. on Biomedical Engineering, vol. 34, pp. 283-88, 1987; and Babiloni and Babiloni, “A high resolution EEG method based on the correction of the surface Laplacian estimate for the subject's variable scalp thickness”, Electroencephalography and Clinical Neurophysiology Volume 103, Issue 4, pp. 486-492, October 1997]. Another such method is an iterative method which solves the Laplace Equation within the volume between the cortex and the scalp while defining cortical potential distribution as excitation. A multidimensional optimization scheme is then employed to provide the potential distribution on the cortical surface [Le and Gevins, “Method to reduce blur distortion from EEGs using a realistic head model”, IEEE Transactions on Biomedical Engineering, Vol. 40, no. 6, June 1993].

### SUMMARY OF THE INVENTION

According to an aspect of some embodiments of the present invention there is provided a method of estimating potential distribution over a cortical surface of a brain of a subject having a head. The method comprises: obtaining encephalogram (EG) data recorded from a scalp surface of the head, and head model data describing a geometry of the head and electrical property distribution of tissues within the head. The method further comprises calculating differentials of the EG data over the scalp surface, and projecting the differentials onto the cortical surface, based on the head model data. The method further comprises calculating volumetric distribution of electrical potential between the cortex and scalp surfaces using the EG data and the projected differentials; and estimating the potential distribution over the cortical surface based on the volumetric distribution.

According to some embodiments of the invention the calculating the volumetric distribution comprises numerically solving a Laplace equation within a volume defined between the surfaces, under boundary conditions defined using the EG data and using the projected differentials.

According to some embodiments of the invention one of the boundary conditions is a Dirichlet boundary condition and another one of the boundary conditions is a Neumann boundary condition.

According to some embodiments of the invention the Dirichlet boundary condition is defined over the scalp surface, and the Neumann boundary condition is defined over the cortical surface.

According to some embodiments of the invention the projection comprises a geometrical mapping of the differentials from the scalp surface onto the cortical surface, irrespectively of the electrical property distribution.

According to some embodiments of the invention the projection comprises a geometrical mapping of the differentials from the scalp surface onto the cortical surface, and an electrodynamic transformation of a value of the differentials based on the electrical property distribution.

According to some embodiments of the invention the differentials comprise surface Laplacian.

According to some embodiments of the invention the EG data comprises processed EG data that is interpolated over the scalp surface.

According to some embodiments of the invention the method comprises processing the EG data to interpolate the EG data over the scalp surface.

According to some embodiments of the invention the invention the method comprises receiving input pertaining to a contact area of EG electrodes and correcting the interpolation based on the contact area.

According to some embodiments of the invention the invention the method comprises estimating displacement of EG electrodes and correcting the interpolation based on the estimated displacement.

According to some embodiments of the invention the method comprises obtaining an image of the head, and constructing the head model from the image to provide the head model data.

According to some embodiments of the invention the image comprises an MRI scan.

According to some embodiments of the invention the constructing the head model comprises segmenting the image according to at least three tissue types, and assigning a predetermined value of the electrical property to each tissue type.

According to some embodiments of the invention the electrical property comprises at least one of electrical conductivity and electrical resistivity.

According to some embodiments of the invention the method comprises displaying on a display device a graphical user interface (GUI) having a head model viewing region showing the head model, a user input control displaying changeable parameters characterizing the head model, and a calculation activation control, and repeating the construction of the head model using parameters in the user input control responsively to an activation of the control and to a change in the parameters.

According to some embodiments of the invention the method comprises calculating a score describing the estimation.

According to some embodiments of the invention the method comprises iteratively repeating the calculation of the volumetric distribution and estimation of the potential distribution over the cortical surface, until the score is within a predetermined score range.

According to some embodiments of the invention the method comprises comparing the estimated potential distribution to a previously estimated potential distribution, and assessing a change in a condition of the subject based on the comparison.

According to some embodiments of the invention the EG data is recorded during and/or after a treatment, and the method comprises comparing the estimated potential distribution to a previously estimated potential distribution, and assessing the effect of the treatment based on the comparison.

According to an aspect of some embodiments of the present invention there is provided a system for estimating potential distribution over a cortical surface of a brain of a subject having a head, the system comprises a data processor configured for receiving encephalogram (EG) data recorded from a scalp surface of the head, and head model data describing a geometry of the head and electrical property distribution of tissues within the head, and executing one or more of the method operations as delineated above, and optionally one or more of the method operations further detailed below.

According to an aspect of some embodiments of the present invention there is provided a computer software product, comprising a computer-readable medium in which program instructions are stored, which instructions, when read by a data processor, cause the data processor to receive encephalogram (EG) data recorded from a scalp surface of the head, and head model data describing a geometry of the head and electrical property distribution of tissues within the head and to execute one or more of the method operations as delineated above, and optionally one or more of the method operations further detailed below.

In some of any of the embodiments described herein a numerical estimation of the scalp surface Laplacian is back-projected onto the cortex to serve as boundary conditions of the cortical normal currents. In some of any of the embodiments described herein the Laplace equation is solved with boundary conditions wrapping the entire solution volume to generate a single and unique solution. In some of any of the embodiments described herein the realistic head model accounts for diploe spongy bone and air cavities as separate layers inside the head volume. These layers can be extracted by a segmentation procedure from an anatomic image of the head.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF SEVERAL VIEWS OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart diagram of a method suitable for estimating potential distribution over a cortical surface of a brain of a subject, according to various exemplary embodiments of the present invention;

FIG. 2 is a method suitable for constructing a head model, according to some embodiments of the present invention;

FIGS. 3A and 3B are screenshots showing a graphical user interface (GUI) that can be used in the method described in FIG. 2, according to some embodiments of the present invention on a display device;

FIG. 4 is a schematic illustration of a system suitable for estimating potential distribution over a cortical surface of a brain of a subject, and optionally also for treating the subject, according to some embodiments of the present invention;

FIG. 5 is a schematic illustration of a process employed in experiments performed according to some embodiments of the present invention;

FIGS. 6A-K show a head model obtained during experiments performed according to some embodiments of the present invention;

FIG. 7A is schematic illustration of a head model composed of three concentric spheres, used in a validation procedure of experiments performed according to some embodiments of the present invention;

FIG. 7B is schematic illustration of dipole structure distributed in the head model shown in FIG. 7B;

FIGS. 7C-E show results of the validation procedure shown in FIGS. 7A and 7B;

FIGS. 8A-C show potential distributions over a realistic head model as obtained during experiments performed according to some embodiments of the present invention;

FIG. 9 is block diagram describing a first channel of validation design process according to some embodiments of the present invention;

FIG. 10 is a block diagram describing second channel of the validation design process according to some embodiments of the present invention;

FIGS. 11A-D illustrate electrodes systems used in experiments performed according to some embodiments of the present invention;

FIGS. 12A-I show sources distributions obtained for BP-CPI validation on realistic head model, according to some embodiments of the present invention;

FIGS. 13A-J depict results of true scalp and cortical potentials associated to the AEP sources, along with BP-CPI results, while adding noise with different power, obtained during experiments performed according to some embodiments of the present invention (for color scale, see FIGS. 17A-J);

FIG. 14 shows quantitative measure of estimation quality as a function of a noise level obtained during experiments performed according to some embodiments of the present invention;

FIGS. 15A-J show scalp potentials sampled with different sets of electrodes, and the corresponding estimated cortical potentials, as obtained during experiments performed according to some embodiments of the present invention (for color scale, see FIGS. 17A-J);

FIGS. 16A-J show electrode displacement error sensitivities for the scalp (FIGS. 16A-E) and the lower skull (FIGS. 16F-J) potentials, for a reference solution (FIGS. 16A and 16F), and four values of a displacement noise: 0 mm (FIGS. 16B and 16G), about 2 mm (FIGS. 16C and 16H), about 4 mm (FIGS. 16D and 16I) and about 14 mm (FIGS. 16E and 16J), as obtained during experiments performed according to some embodiments of the present invention (for color scale, see FIGS. 17A-J);

FIG. 16K is a graph showing a calculated Pearson's correlation coefficient as a function of electrode displacement standard deviation (STD), as obtained during experiments performed according to some embodiments of the present invention;

FIGS. 17A-J show conductivity estimation sensitivities for the scalp (FIGS. 17A-E) and cortex (FIGS. 17F-J) potentials, as obtained using 128 EEG electrodes during experiments performed according to some embodiments of the present invention;

FIGS. 18A-I show sensitivities to the depth of the source, as obtained during experiments performed according to some embodiments of the present invention;

FIGS. 19A-I show sensitivities to the spread of the sources, as obtained during experiments performed according to some embodiments of the present invention;

FIGS. 20A-C show EEG electrode alignment employed during experiments performed according to some embodiments of the present invention;

FIGS. 21A-C show forward solution validation as obtained during experiments performed according to some embodiments of the present invention;

FIG. 22 shows current distribution and components involved in a back projection procedure employed according to some embodiments of the present invention;

FIGS. 23A-D show results obtained during experiments performed according to some embodiments of the present invention for the validation of a surface Laplacian as a cortical estimator;

FIGS. 24A-D show sources orientation and location used for BP-CPI validation (FIG. 24A), analytical forward solutions on scalp (FIG. 24B) and cortical (FIG. 24C) surfaces, and estimated cortical potentials (FIG. 24D);

FIGS. 25A-F show axial, coronal and sagittal views of source distributions for BP-CPI validation on realistic head model, incorporating seven visual evoked potential (VEP) (FIGS. 25A-C) and two auditory evoked potential (AEP) (FIGS. 25D-F) source locations and orientations;

FIGS. 26A-F show forward solution results obtained according to some embodiments of the present invention for VEP and AEP source distributions;

FIGS. 27A-L show grand average and single-subject event-related potentials obtained from EEG data collected during experiments performed according to some embodiments of the present invention;

FIGS. 28A-F show fMRI slices obtained during experiments performed according to some embodiments of the present invention;

FIGS. 29A-F show cortical potential maps obtained according to some embodiments of the present invention from EEG data collected simultaneously during acquisition of fMRI scans; and

FIGS. 30A-F show the cortical fMRI activation shown in FIGS. 28A-F, superimposed on a three-dimensional synthetic brain model.

### DESCRIPTION OF SPECIFIC EMBODIMENTS OF THE INVENTION

The present invention, in some embodiments thereof, relates to medical imaging and, more particularly, but not exclusively, to a method and system for generating an image describing an estimate of the potential distribution on the cortical surface.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Embodiments of the present invention are directed to a technique for estimating potential distribution on a surface using electrical potential measured on another surface, and the electrical property distribution and geometry of a volume between the two surfaces. In any of the embodiments described herein, the surface is preferably the cortical surface of a brain of a subject, e.g., a mammalian subject, preferably a human subject. Optionally, but not necessarily, the estimated potential distribution is thereafter used for assessing a change in a condition of the brain and/or the effect of a particular treatment applied to the subject.

It is to be understood that, unless otherwise defined, the operations described hereinbelow can be executed either contemporaneously or sequentially in many combinations or orders of execution. Specifically, the ordering of the flowchart diagrams is not to be considered as limiting. For example, two or more operations, appearing in the following description or in the flowchart diagrams in a particular order, can be executed in a different order (e.g., a reverse order) or substantially contemporaneously. Additionally, several operations described below are optional and may not be executed.

At least part of the operations can be can be implemented by a data processing system, e.g., a dedicated circuitry or a general purpose computer, configured for receiving the data and executing the operations described below. At least part of the operations can be can be implemented by a cloud-computing facility at a remote location.

Computer programs implementing the method of the present embodiments can commonly be distributed to users on a distribution medium such as, but not limited to, a floppy disk, a CD-ROM, a flash memory device and a portable hard drive. From the distribution medium, the computer programs can be copied to a hard disk or a similar intermediate storage medium. The computer programs can be run by loading the computer instructions either from their distribution medium or their intermediate storage medium into the execution memory of the computer, configuring the computer to act in accordance with the method of this invention. All these operations are well-known to those skilled in the art of computer systems.

The method of the present embodiments can be embodied in many forms. For example, it can be embodied in on a tangible medium such as a computer for performing the method operations. It can be embodied on a computer readable medium, comprising computer readable instructions for carrying out the method operations. In can also be embodied in electronic device having digital computer capabilities arranged to run the computer program on the tangible medium or execute the instruction on a computer readable medium.

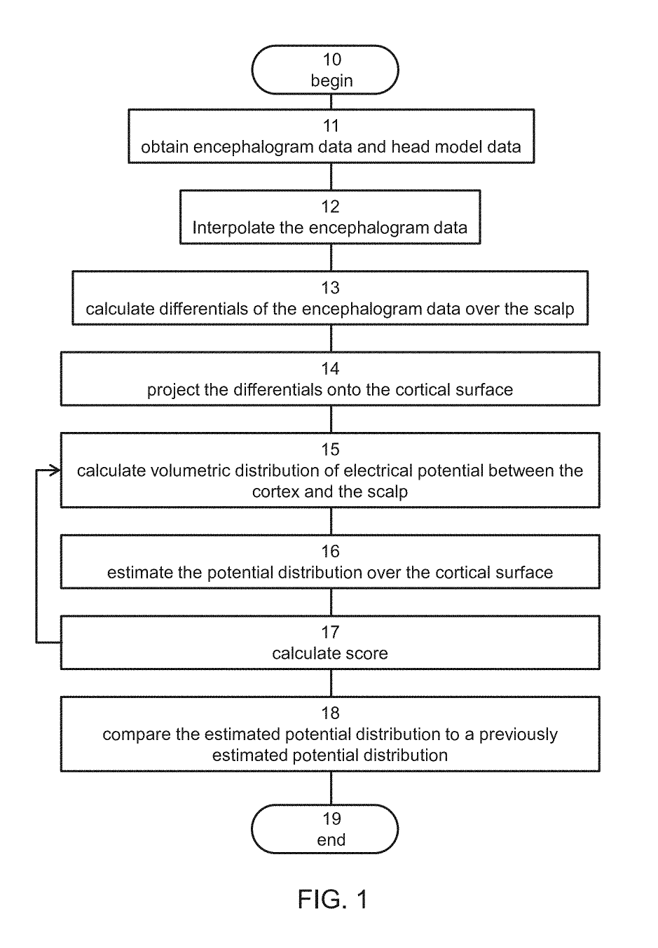

Reference is now made to FIG. 1 which is a flowchart diagram of a method suitable for estimating potential distribution over a cortical surface of a brain of a subject, according to various exemplary embodiments of the present invention.

The method begins at 10 and continues to 11 at which encephalogram (EG) data and head model data are obtained. In any of the embodiments described herein, the EG data can include electroencephalogram data (EEG data), magnetoencephalogram data (MEG data), both EEG data and MEG data, a combination (e.g., an average, a weighted average) of EEG data and MEG data normalized to allow such combination or a selective local substitution of either EEG data or MEG data based on some criterion or set of criteria, following for example, a statistical analysis of the EEG data or MEG data at each measuring location.

The EG data are recorded from a scalp surface of the subject's head, and can include a plurality of waveforms, which are typically time domain waveforms, where each waveform corresponds to a different EG channel and describes electrical potentials measured at a different location over the scalp. One or more of the waveforms, preferably all the waveforms can also be decomposed into a plurality of partial waveforms each corresponding to different frequency range within the waveform. The EG data can be either received from an external source (for example, a data storage system storing the EG data, optionally and preferably in a digitized form, on a suitable storage medium), or it can be measured by the method using an EG system having EG electrodes connected to the scalp, and an EG measuring device that receives electrical signals from the electrodes and converts the signals to EG data, optionally and preferably digitized EG data.

The head model data describe geometric properties of the head and electrical property distribution of tissues within the head. Optionally, the head model data also include data pertaining to air cavities sparsely located within the head.

The geometric properties are typically in the form of a three-dimensional (3D) spatial representation defining a non-planar surface, and a 3D volume enclosed by this non-planar surface. The non-planar surface can represent the scalp and the three-dimensional volume can represent the interior of the head. The volume is preferably, but not necessarily, a volumetric shell within the interior of the head, e.g., a shell having an inner surface corresponding to the cortical surface, and an outer surface corresponding to the scalp surface. Generally, the non-planar surface is a two-dimensional (2D) object embedded in a 3D space. The surface and enclosed volume (e.g., volumetric shell) is typically represented by a plurality of volume elements that can be defined over a point-cloud or, more preferably a 3D reconstruction (e.g., a polygonal mesh or a curvilinear mesh) based on the point cloud. The 3D spatial representation is expressed via a 3D coordinate system, such as, but not limited to, Cartesian, Spherical, Ellipsoidal, 3D Parabolic or Paraboloidal 3D coordinate system.

The electrical property distribution comprises values of the electrical property for each of at least portion of the volume elements that compose the 3D spatial representation. The value of the electrical property for a particular volume element is the characteristic electrical property of tissue that the particular volume element describes within the head. The electrical property is typically an intrinsic electrical property, such as, but not limited to, conductivity or resistivity. Typically, the electrical property value of a particular volume element is one of a set of possible predetermined values that are characteristic to tissue types within the head. Preferably, the set includes at least three or at least four different values.

In some embodiments, the set can include three different values corresponding to scalp tissue, compact bone and spongy bone. As a representative example for such a three-value set, in embodiments in which the electrical property is conductivity, the conductivity corresponding to scalp tissue can be from about 0.3 to about 0.36, e.g., about 0.33 S/m, the conductivity corresponding to compact bone can be from about 0.0039 to about 0.0045, e.g., about 0.0042 S/m, and the conductivity corresponding to spongy bone can be from about 0.028 to about 0.029, e.g., about 0.0286 S/m. A corresponding three-value set can also be defined in embodiments in which the electrical property is resistivity, using the relation p=1/o, where p is the resistivity and σ is the conductivity. Thus, the resistivity of corresponding to scalp tissue can be from about 2.78 to about 3.33, e.g., about 3.03 Ω·m, the resistivity corresponding to compact bone can be from about 222 to about 256, e.g., about 238 Ω·m, and the resistivity corresponding to spongy bone can be from about 34.48 to about 35.7, e.g., about 34.97 Ω·m. For any given type of tissue, the electrical property distribution can be either homogenous or non-homogenous and either isotropic or non-isotropic across a region occupied by that type of tissue, preferably within a range of electrical property values that are characteristic to that type of tissue.

The electrical property is optionally and preferably assigned to the volume elements in a layerwise manner, such that all the volume elements that define the same layer within the volume (e.g., volumetric shell) of the 3D spatial representation are assigned with the same electrical property. Thus, for example, a layerwise distribution of the electrical property can include an outermost layer that corresponds to the scalp and that is assigned with an electrical property that is characteristic to scalp tissue, an inner layer that corresponds to the higher skull and that is assigned with an electrical property that is characteristic to compact bone, another inner layer that corresponds to the intermediate skull and that is assigned with an electrical property that is characteristic to spongy bone, and a innermost layer that corresponds to the lower skull and that is also assigned with an electrical property that is characteristic to compact bone.

The head model data can optionally be in the form of a synthesized 3D image which includes both the electrical property distribution and the 3D spatial representation on the same 3D image. Such image is referred to as a head model image.

The head model can be either received from an external source (for example, a data storage system storing the head model data, optionally and preferably in a digitized form, on a suitable storage medium), or it can be generated by the method, for example, by processing an image, e.g., a magnetic resonance image (MRI), of the head. A representative example of a technique suitable for generating a head model suitable for the present embodiments is provided hereinbelow.

When the method relieves the data (EG data and/or head model data) from an external source, the external source can be local or remote. For example, the method can access a local storage medium for obtaining the data, or it can download the data via a communication network (e.g., the internet) from a remote storage medium (e.g., a cloud storage facility).

The method optionally and preferably continues to 12 at which the EG data is processed to interpolate the EG data over scalp surface, hence to provide interpolated EG data. The interpolation typically provides scalp potentials also at locations of the scalp at which no EG electrode was connected. The interpolation can be polynomial interpolation, using a first-order polynomial function (a linear function), a second-order polynomial function (a quadratic function), a third-order polynomial function (a cubic function) or any polynomial function of degree n where n≥1. The interpolation can also employ non-linear functions that are not necessarily polynomial, for example, logarithmic functions or exponential functions.

While the interpolation can, in principle, provides EG data that vary continuously over the scalp surface, this need not necessary be the case. Preferably, the interpolation provides scalp potentials at each of a plurality of discrete points over the scalp surface to provide spatial resolution that is compatible with the spatial resolution of the head model. For example, the interpolation can provide a scalp potential at each of the vertices of the 3D reconstruction that form the non-planar surface of the 3D spatial representation. Preferably, the method receives input pertaining to a contact area of the EG electrodes and corrects the interpolation based on the contact area. Also contemplated are embodiments in which displacement of the EG electrodes is estimated and the interpolation is corrected based on the estimated displacement.

The method optionally and preferably continues to 13 at which differentials of EG data, for example, of scalp potentials, are calculated over the scalp surface. The differentials are preferably calculated using second-order partial derivatives. The differentials are preferably calculated at each of at least some of the vertices of the 3D reconstruction that form the non-planar surface of the 3D spatial representation. In some embodiments, all the vertices of the 3D spatial representation are visited and the differentials are calculated at each vertex, and in some embodiments only varices that belong to a subset of all the vertices of the 3D spatial representation are visited and the differentials are calculated only for the varices of the subset. The subset can be selected randomly or according to some criterion or set of criteria. As a representative example which is not to be considered as limiting, the subset can include varices that correspond to the location of the EG electrodes and optionally and preferably also vertices that are nearby those vertices. In some embodiments of the present invention, for each such vertex, a parametric surface, preferably a non-planar surface is defined in the vicinity of the vertex using a plurality of vertices nearby that vertex. The differentials can then be calculated by solving a set of equations, optionally and preferably a linear set of equations, defined over the parametric surface. The set of equations can be formally written as a matrix equation:

Ad=v

where A is a matrix of discrete spatial differentials over the parametric surface, d is a vector of derivatives of a surface potential function V over the parametric surface, and v is a vector of discrete surface potential function differentials over the parametric surface.

Formally, denoting the Cartesian coordinates of a particular vertex by (x₀,y₀,z₀), the parametric surface can be represented in a parametric 2D space defined over coordinates (ξ,η) such that x=f(ξ,η), y=g(ξ,η) and z=h(ξ,η), wherein the vertex (x₀,y₀,z₀) is mapped onto the 2D space at (ξ₀,η₀). This parametric surface can be used to define a surface potential and to calculate differentials of the scalp potential at (ξ₀,η₀). Denoting the scalp potentials of the EG data over the 3D Cartesian space by the potential function U(x,y,z), the surface potential function V can be defined over the parametric surface based on the potential function U using the relation:

U(x,y,z)=U(f(ξ,η),g(ξ,η),h(ξ,η))=V(ξ,η).

Once the surface potential function V(ξ,η) is defined, its differentials with respect to the surface coordinates ξ and η can be calculated at (ξ₀,η₀). In any of the embodiments of the present invention the differentials comprise a surface Laplacian. A surface Laplacian ∇²V of the surface potential V in Cartesian coordinates is typically defined as:

$${∇}^{2} V = \frac{{\partial }^{2} V}{\partial {ξ}^{2}} + \frac{{\partial }^{2} V}{\partial {η}^{2}} ,$$

where ∂²V/∂ξ² and ∂²V/∂η² are second derivatives of V. The derivatives are preferably estimated at vertex (ξ₀,η₀) using the aforementioned set of equations. The set of equations can be defined by expanding V at the vicinity of (ξ₀,η₀) using on the value of V at (ξ₀,η₀) and at the vertices nearby (ξ₀,η₀). The expansion of V using the ith vertex that is in the vicinity of the vertex (ξ₀,η₀), can be written as:

$$V ( {ξ}_{i} , {η}_{i} ) - V ( {ξ}_{0} , {η}_{0} ) \approx \frac{\partial V ( {ξ}_{0} , {η}_{0} )}{\partial ξ} ( {ξ}_{i} - {ξ}_{0} ) + \frac{\partial V ( {ξ}_{0} , {η}_{0} )}{\partial η} ( {η}_{i} - {η}_{0} ) + \frac{1}{2} \frac{{\partial }^{2} V ( {ξ}_{0} , {η}_{0} )}{\partial {ξ}^{2}} {( {ξ}_{i} - {ξ}_{0} )}^{2} + \frac{1}{2} \frac{{\partial }^{2} V ( {ξ}_{0} , {η}_{0} )}{\partial {η}^{2}} {( {η}_{i} - {η}_{0} )}^{2} + \frac{{\partial }^{2} V ( {ξ}_{0} , {η}_{0} )}{\partial η \partial ξ} ( {η}_{i} - {η}_{0} ) ( {ξ}_{i} - {ξ}_{0} ) ,$$

where (ξᵢ,ηᵢ) are the coordinates over the parametric surface of the ith vertex.

The number of vertices at the vicinity of (ξ₀,η₀) that are selected to obtain the derivatives ∂²V/∂ξ² and ∂²V/∂η² is at least the number of terms in the expansion of V. In the present example, the number of vertices is at least 5, but more than five vertices (e.g., 6, 7, 8, 9, 10, 11, 12, 13, 14, 15, 16, 17, 18, 19, 20 or more vertices) are preferably selected to improve accuracy.

When the above expansion is used, the matrix A, and vectors d and v can be written as:

$$A = [ \begin{matrix}\frac{1}{2} {( {ξ}_{1} - {ξ}_{0} )}^{2} & \frac{1}{2} {( {η}_{1} - {η}_{0} )}^{2} & ( {η}_{1} - {η}_{0} ) ( {ξ}_{1} - {ξ}_{0} ) & ( {ξ}_{1} - {ξ}_{0} ) ( {η}_{1} - {η}_{0} ) \\ \cdot & \cdot & \cdot & \cdot \cdot \\ M & M & M & M M \\ \frac{1}{2} {( {ξ}_{k} - {ξ}_{0} )}^{2} & \frac{1}{2} {( {η}_{k} - {η}_{0} )}^{2} & ( {η}_{k} - {η}_{0} ) ( {ξ}_{k} - {ξ}_{0} ) & ( {ξ}_{k} - {ξ}_{0} ) ( {η}_{k} - {η}_{0} )\end{matrix} ] \underline{d} = [ \begin{matrix}\frac{{\partial }^{2} V ( {ξ}_{0} , {η}_{0} )}{\partial {ξ}^{2}} \\ \frac{{\partial }^{2} V ( {ξ}_{0} , {η}_{0} )}{\partial {η}^{2}} \\ \frac{{\partial }^{2} V ( {ξ}_{0} , {η}_{0} )}{\partial η \partial ξ} \\ \frac{\partial V ( {ξ}_{0} , {η}_{0} )}{\partial ξ} \\ \frac{\partial V ( {ξ}_{0} , {η}_{0} )}{\partial η}\end{matrix} ] and \underline{v} = [ \begin{matrix}V ( {ξ}_{1} , {η}_{12} ) - V ( {ξ}_{0} - {η}_{0} ) \\ M \\ V ( {ξ}_{k} , η k ) - V ( {ξ}_{0} , {η}_{0} )\end{matrix} ]$$

where k is the number of vertices at the vicinity of (ξ₀,η₀) that are used.

While the vector d above includes five components, it is appreciated that it is not necessary to explicitly calculate and store for further usage each of these five components (although such explicit calculation is also contemplated). For example, when it is desired to determine only the surface Laplacian ∇²V, it sufficient to calculate and store only the first two components of d, since ∇²V(ξ₀,η₀)=d₁+d₂.

In some embodiments of the present invention 13 is accompanied by application of one or more smoothing filters (e.g., low pass Gaussian filters) before and/or after the calculation of the differentials. The width of the smoothing filter(s) is optionally and preferably determined based on the head model data, and can be either subject-specific or defined per group of subjects (for example, a group of subjects having similar head geometry).

Once the differentials are calculated, the method optionally and preferably continues to 14 at which the differentials are projected onto the cortical surface. This can be done based on the head model data. Typically, scalp surface coordinates within the 3D spatial representation are geometrically mapped to cortical surface coordinates within the 3D spatial representation, by projecting a plurality (e.g., at least 10 or at least 15 or at least 20 or more) of arbitrarily selected positions on the scalp surface onto positions on the cortical surface underlying the positions on the scalp surface, and determining the coordinate positions of the projected points. Thereafter, the value of the differential at each arbitrarily selected position on the scalp surface is assigned to the respective projected position on the on the cortical surface.

In some embodiments of the present invention, the projection of the differentials comprises both geometrical mapping and electrodynamic transformation of the value of the differentials, where the electrodynamic transformation is based on the distribution of the electrical property between the scalp surface and the cortical surface. In these embodiments, for each determined coordinate position of a projected point, the value of the differential is electrodynamically transformed from an obtained value at the respective point on the scalp surface to a transformed value at the projected point. The transformed value is calculated by calculating the change in the differential due to the change of the electrical property structure along a path passing from the point on the scalp surface to the respective point on the cortical surface, through the multilayer distribution of the electrical property structure between the surfaces.

The geometrical mapping of scalp surface coordinates to cortical surface coordinates can be done in more than one way. In some embodiments of the present invention the mapping employs a minimum distance search method, in some embodiments of the present invention the mapping employs a perpendicular projection method, in some embodiments of the present invention the mapping employs segment connecting method, and in some embodiments of the present invention the mapping employs electrical path selection method.

In the minimum search method, equidistant spheres each having a different radius from an arbitrary point on a scalp surface are defined, and contact points of the spheres and the cortical surface are determined. When the scalp surface is in the vicinity of a protrusion in the cortical surface, one point having the minimum distance can be determined. When the scalp surface is positioned in the vicinity of a depression of the cortical surface, two or more points can be determined. In the latter case, the center of gravity of these points is supposed to be the virtual minimum point, a straight line passing through the virtual minimum point from the scalp surface point in question is defined, and an intersection point of the straight line and the cortical surface is defined as a point underlying the scalp surface point in question.

In the perpendicular projection method, a plane being in contact with an arbitrary point on the scalp surface is defined, and a vertical line is defined downwards from the contact point on the plane to the cortical surface, whereby an intersection point with the cortical surface is determined as a projection point. When there is no mesh point in that location, the closest point which is defined by the mesh is of the present embodiments selected.

In the scalp segment connecting method, a straight line is defined from an arbitrary point on the scalp surface to a reference point inside the brain surrounded by the scalp surface, and an intersection point of the straight line and the cortical surface is determined as a projection point for the scalp surface point in question. The reference point inside the brain can be an arbitrary point or a set of points. For example, a weighted center point of the scalp surface or the cortical surface can be defined as the reference point. Alternatively, the reference point can be a specific brain structure such as, but not limited to, an anterior commissure. Also contemplated are embodiments in which the reference point is the weighted center point on the scalp surface in the vicinity centering around an arbitrary point on the scalp surface, and the point on the scalp surface in question is deviated to obtain a set of reference points.

In the electrical path selection method, a set of expected current trajectories within the volumetric shell is determined based on the electrical property values of the volume elements that form the shell. This set can be filtered using a probabilistic function, wherein current trajectories along which the accumulated electrical resistance is higher are suppressed relative to current trajectories along which the accumulated electrical resistance is lower. Once a set of expected current trajectories is obtained, the mapping of scalp surface coordinates to cortical surface coordinates is done based on these trajectories. Specifically, a point or a region over the cortical surface is defined as the projection of a point or a region over the scalp surface if there is a current trajectory within the set that connects between these points.

In some embodiments the method projects the potentials as obtained from the EG data onto the cortical surface. This can be done as described above with respect to the differentials except that the projected entity at each point over the scalp surface is the potential rather than the surface differential. The potentials can be projected either instead of the differentials or in addition to the differentials.

The method continues to 15 at which volumetric distribution of electrical potential is calculated between the cortical surface and the scalp surface. This calculation is based on the EG data, preferably on the electrical potential over the scalp surface. This calculation can also be based on the projected differentials and/or the projected potentials. For example, a partial differential equation, preferably a second-order partial differential equation, for example, the Laplace equation, can be numerically solved within the volumetric shell defined between the two surfaces, under boundary conditions defined using the EG data and using the projected differentials.

For any embodiment of the invention, a preferred implementation is that the partial differential equation is solved, at least once, without any input or assumption regarding the values of the electrical potential at the cortical surface. A solution in which the values of the electrical potential at the cortical surface are assumed to be know is referred to as a “solution to the forward problem” or, more concisely, a “forward solution,” and a solution in which the values of the electrical potential at the cortical surface are not assumed to be know is referred to as a “solution to the backward problem” or, more concisely, a “backward solution”. Thus, a preferred implementation of any embodiment of the invention is to obtain, at least one, a solution to the backward problem within the volumetric shell defined between the cortical surface and the scalp surface.

Typically, one of the boundary conditions for the partial differential equation is a Dirichlet boundary condition and another boundary conditions is a Neumann boundary condition. The Dirichlet boundary condition is preferably defined over the scalp, in which case the value of the volumetric distribution of the electrical potential at the scalp surface is set as the electrical potential as obtained from the EG data. The Neumann boundary condition is preferably defined over the cortical surface, in which case the normal directional derivative of the volumetric distribution of the electrical potential over the cortical surface is set as the projected differentials. Thus, the present embodiments contemplate an implementation in which the partial differential equation is solved, at least once, using the derivative but not the value of the electrical potential at the cortical surface.

In alternative embodiment, the potentials are projected onto the cortical surface. In these embodiments a Dirichlet boundary condition can defined over the cortex, in which case the value of the volumetric distribution of the electrical potential at the cortical surface is set as the electrical potential as projected, and a Dirichlet boundary condition or a Neumann boundary condition can be defined over the scalp. In the former case (Dirichlet boundary condition over the scalp surface) the value of the volumetric distribution of the electrical potential at the scalp surface is set as the electrical potential as obtained from the EG data. In the latter case (Neumann boundary condition over the scalp surface), the normal directional derivative of the volumetric distribution of the electrical potential over the scalp surface is set as the differentials calculated at 13.

The solution of the partial differential equation can be obtained by computer simulations, for example, using a numerical finite element method, discrete element method, finite difference method, finite boundary analysis, etc. This can be done using commercial software, such as ANSYS®, MATLAB®, Sim4Life® or the like. A representative example of a finite element method suitable for the present embodiments is provided in the Examples section that follows.

The method can then continue to 16 at which the potential distribution over the cortical surface is estimated. This is preferably done by evaluating volumetric distribution values at each of a plurality of points over the cortical surface, using the calculated volumetric distribution, and defining the estimated potential distribution to be equal to the evaluated values. In some embodiments of the present invention the method continues to 17 at which the method calculates a score of the estimated potential distribution. This can be done, for example, by solving the partial differential equation wherein both a Dirichlet and a Neumann boundary conditions are imposed at the cortical surface, using the solution for calculating the value of the electrical potential over the scalp surface, comparing the electrical potential as obtained from the EG data to the calculated electrical potential over the scalp surface, and using this comparison as a basis for the score. A representative example of an iterative procedure according to some embodiments of the present invention is as follows. In each iteration, result for the cortical potentials as obtained in the previous iteration is varied, randomly or according to a predetermined criterion. The scalp potentials that are a solution to the forward problem are then calculated, using the varied cortical potentials as the Dirichlet boundary condition on the cortical surface. The forward-calculated scalp potentials are then compared to the EG data at the electrodes, and a fitting score is optionally and preferably calculated based on this comparison. The fitting score can be, for example, a correlation coefficient, a relative error (e.g., mean square error), a covariance matrix, a maximal difference, an area between potential plots, an Euclidean distance, a Mahalanobis distance, an Lp-norms and the like. Optionally, a stability score is also calculated for the background and/or forward solution of each iteration.

The method can optionally and preferably loop back to 15 and repeat the calculation until the score or scores are within a predetermined score range. The repetition is optionally and preferably in an iterative manner, wherein in each repetition, the previously estimation of the potential distribution over the cortical surface of is used for constructing the boundary condition. In these embodiments, the boundary condition at the cortical surface can be either a Dirichlet or a Neumann boundary condition, and the boundary condition at the scalp surface is preferably a Dirichlet boundary condition as in the first iteration.

In some embodiments of the present invention the method continues to 18 at which the estimated potential distribution is compared to a previously estimated potential distribution, preferably of the same subject, but may also be a library potential distribution. Such a comparison can be used in more than one way. In some embodiments, the comparison is used for assessing a change in the brain condition or brain function of the subject. In some embodiments, the previously estimated potential distribution corresponds to EG data rescored before treatment and the currently estimated potential distribution corresponds to EG data recorded during and/or after a treatment. In these embodiments the comparison is used for assessing the effect of the treatment on the brain of the subject. For example, the comparison may be indicative of whether or not the subject is responsive to the treatment and, in some examples, whether the treatment is expected to be effective at treating the condition of the patient. For example, when electrical stimulation of the brain increases the cortical potentials, the electrical stimulation can be determined as effective at treating the condition. In some examples, the difference between the cortical potentials before and after the treatment is determined, wherein a difference that is greater than an efficacy threshold indicates that the subject is responsive to the treatment and, in some examples, indicate that the treatment may be effective in reducing or eliminating the symptoms of the subject's condition.

In any of the above embodiments, when the estimated cortical potential is compared to the previously estimated cortical potential, one or more characteristics of the cortical potential may be compared. In other words, comparison of cortical potential may include comparing respective values of a common characteristic that at least partially describes the respective potential.

Also contemplated, are embodiments in which a plurality of different treatment parameter sets are evaluated to identify an effective treatment. For example, several treatment sessions can be applied, each time with a different parameter sets. Following each session, the effect of the treatment can be assessed as further detailed hereinabove, and the method can select the parameter set that induced the largest change in the cortical potential.

Further contemplated are embodiments in which the parameter set is varied within the same treatment session, and the method indicates when a particular parameter set variation induce a detectable change or the largest detectable change in the cortical potential

As used herein, the term “treatment” includes abrogating, substantially inhibiting, slowing or reversing the progression of a condition, substantially ameliorating clinical or aesthetical symptoms of a condition or substantially preventing the appearance of clinical or aesthetical symptoms of a condition. Treatment can include any type of intervention, both invasive and noninvasive, including, without limitation, pharmacological, surgical, irradiative, rehabilitative, and the like.

The present embodiments contemplate many types of treatments.

In some embodiments, local brain stimulation is employed by a local brain stimulation tool. Typically, the local brain stimulation tool is configured to apply stimulation in pulses and not in a continuous wave (CW) mode.

The tool can apply either non-invasive or invasive stimulations.

Representative examples of types of non-invasive local brain stimulations suitable for the present embodiments include, without limitation transcranial magnetic stimulation (TMS), Transcranial Electrical Stimulation (tES), focused ultrasound stimulation (FUS) and electroconvulsive therapy (ECT).

Representative examples of types of transcranial magnetic stimulations suitable for the present embodiments include, without limitation, repetitive Transcranial Magnetic Stimulation (rTMS), deep Transcranial magnetic stimulation (dTMS), multichannel TMS and multichannel dTMS. Representative examples of types of transcranial electrical stimulations suitable for the present embodiments include, without limitation, Transcranial direct current stimulation (tDCS), Transcranial alternate current stimulation (tACS), Transcranial random noise stimulation (tRNS), High definition tES (HD-tES), High definition tDCS (HD-tDCS), and multichannel tES. Also contemplated are optical stimulations, such as, but not limited to, transcranial infrared laser stimulation or the like.

Representative examples of types of invasive local brain stimulations suitable for the present embodiments include, without limitation electrical invasive stimulation, such as, but not limited to, Deep brain stimulation (DBS) and multifocal DBS.

tES can be either multi-focal or single focal. tES can be employed using any number of electrodes. Typically, the number of electrodes is from 1 to 256, but use of more than 256 electrodes is also contemplated in some embodiments of the present invention.

tDCS and HD-tDCS suitable for the present embodiments are found for example, in Edwards et al., NeuroImage 74 (2013) 266-275; Kuo et al., Brain Stimulation, Volume 6, Issue 4 (2013) 644-648; and Villamar et al., J Pain. (2013) 14(4):371-83, the contents of which are hereby incorporated by reference.

Also contemplated is invasive or no-invasive stimulation by a laser beam, as described, for example, in U.S. Pat. Nos. 8,498,708 and 8,506,613, and combination of any of the above stimulations with invasive or no-invasive stimulation by a laser beam.

The present embodiments also contemplate applying other types of treatments, including, without limitation, hyperbaric therapy, phototherapy, and pharmacological therapy.

Phototherapy can be accomplished by radiating light energy into a subject's tissue at or below the skin or surface of the tissue. The radiation is applied at wavelengths either in the visible range or the invisible infrared (IR) range. Phototherapy may also be accomplished by applying coherent and non-coherent light energy, lased and non-lased light energy, and narrow and broadband light energy, in either a continuous or pulsed manner. The radiation energy is also typically applied at a low power intensity, typically measured in milliwatts. The relatively low radiation energy applied in therapy is called low level light therapy (LLLT). LLLT has also been suggested for neurological disorders in the CNS, for the prevention and/or repair of damage, relief of symptoms, slowing of disease progression, and correction of genetic abnormalities. In particular, phototherapy can be used following a cerebrovascular accident (stroke).

Hyperbaric therapy can be accomplished in a hyperbaric chamber, wherein is provided by administering oxygen to the user via a closed-circuit mask, hood, or other device while a hyperbaric chamber is maintained at pressures above ambient pressure. The oxygen is supplied to the user from a supply source external to the chamber.

Pharmacological therapy can be achieved by a variety of chemical or biological substances, including, for example, the use of a pharmacologically active agent, e.g., centrally acting drugs, particularly CNS active agents and other nervous system agents.

The method ends at 19.

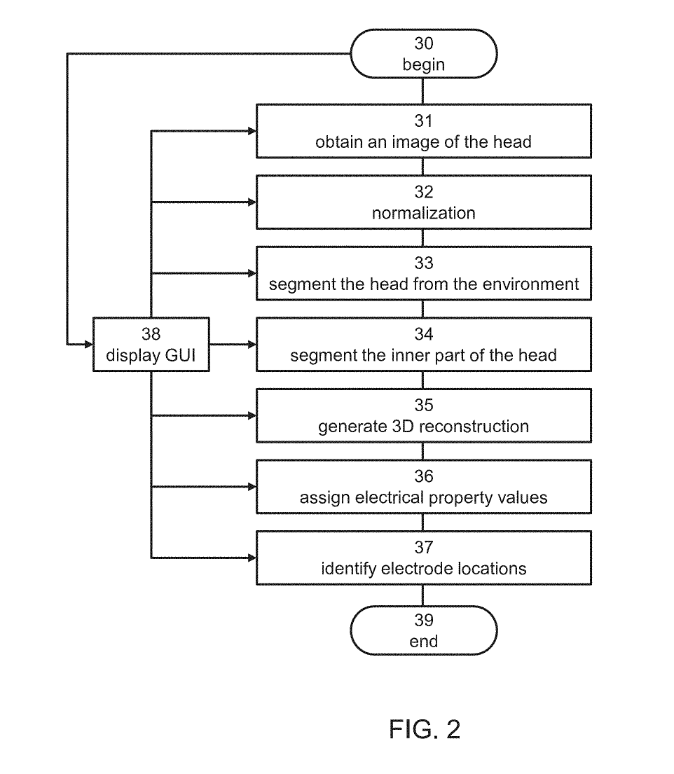

FIG. 2 is a flowchart diagram describing a method suitable for constructing a head model, according to some embodiments of the present invention. The method begins at 30 and optionally and preferably continues to 31 at which an anatomic image, preferably a three-dimensional or sliced image, of the head is obtained. In some embodiments of the present invention the image is an MRI (e.g., a T1-weighted MRI, a T2-weighted MRI, a diffusion-weighted MRI), but other types of images, such as, but not limited to, ultrasound images, computerize tomography (CT) image, positron emission tomography (PET) image, single photon emission computerized tomography (SPECT) and the like.

The method optionally and preferably continues to 32 at which the image normalization is applied. The advantage of using image normalization is because it allows comparing different distribution of the electrical activity from differently shaped heads. The normalization scheme dependents on the type of the image. For example, when the image is an MRI, the normalization includes mapping the image onto the Montreal Neurological Institute (MNI) coordinates.

At 33 a segmentation procedure is employed so as to remove from the image regions that correspond to the environment that surrounds the head. Alternatively, the image can already be provided without the surrounding environment in which case 33 can be skipped. At 34 the inner part of the head is segmented so as to identify different types of tissues therein. Preferably, the segmentation 34 identifies scalp tissue, compact bone, spongy bone and brain tissue. For example, brain tissue can be identified by a commercially available procedure known as brain extraction tool (BET), an add-on applicable for use with the MATLAB® software; and compact bone, cerebrospinal fluid and air cavities can be identified by a commercially available procedure known as statistical parametric mapping (SPM) which is another add-on applicable for use with the MATLAB® software. Spongy tissue can be identified by scanning an MRI with sufficient resolution to distinguish the spongy bone from the other layers. Optionally, an image smoothing operation is applied once the image is segmented.

The method optionally and preferably continues to 35 at which a 3D reconstruction (e.g., a polygonal mesh or a curvilinear mesh) is generated. This can be done by considering the voxels of the image as a point cloud, defining mesh lines between neighboring points, and volume elements (e.g., tetrahedral elements, hexahedral elements) according to the mesh lines. At 36 electrical property values are assigned to the volume elements according to the identified segments. For example, different values of the electrical property can be assigned to volume elements belonging to segments identified as scalp tissue, compact bone and spongy bone either homogenously or non-homogenously and either isotropically or non-isotropically as further detailed hereinabove.

The method can optionally and preferably continue to 37 at which volume elements on the scalp that correspond to the location of the EG electrodes are identified and marked. This can be done in more than one way. In some embodiments of the present invention the MNI coordinates of the electrodes are received from an external source (for example, an EG system or user input), and the points of the 3D reconstruction on the scalp that are closest to the received MNI coordinates are then used as pointers to the volume elements on the scalp that correspond to the location of the electrodes. In some embodiments, the head is imaged (for example, by MRI) while the electrodes are placed on the subject's scalp, and the location of the electrodes are extracted from the image of the head. In these embodiments, a marker that is detectable by the imaging modality is optionally and preferably attached to each electrode so as to facilitate its detection. The points of the 3D reconstruction on the scalp that are closest to the extracted location of the electrodes are then used as pointers to the volume elements on the scalp that correspond to the location of the electrodes.

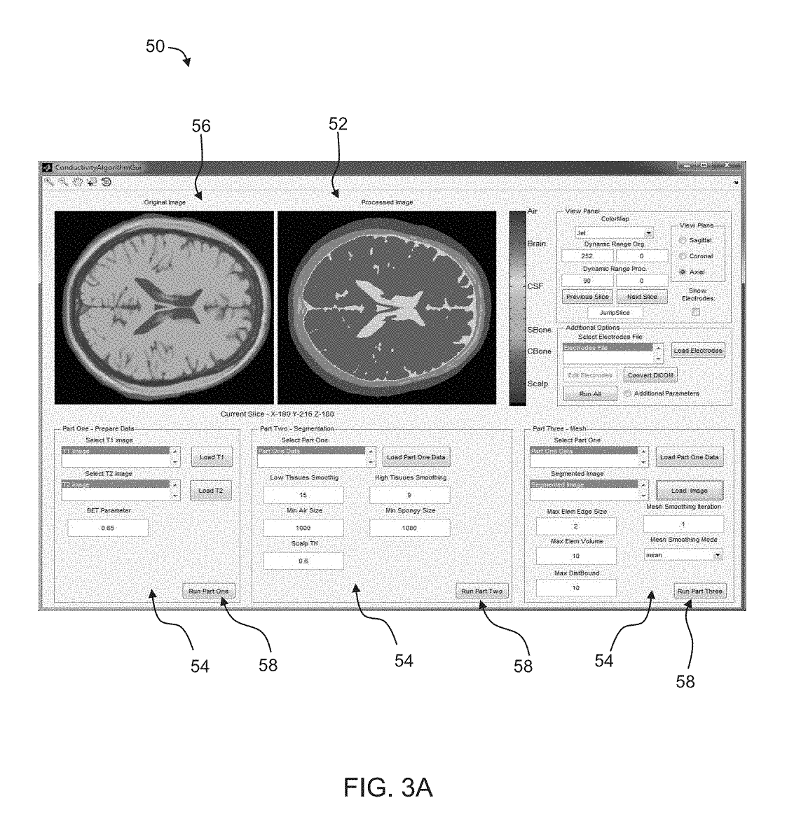

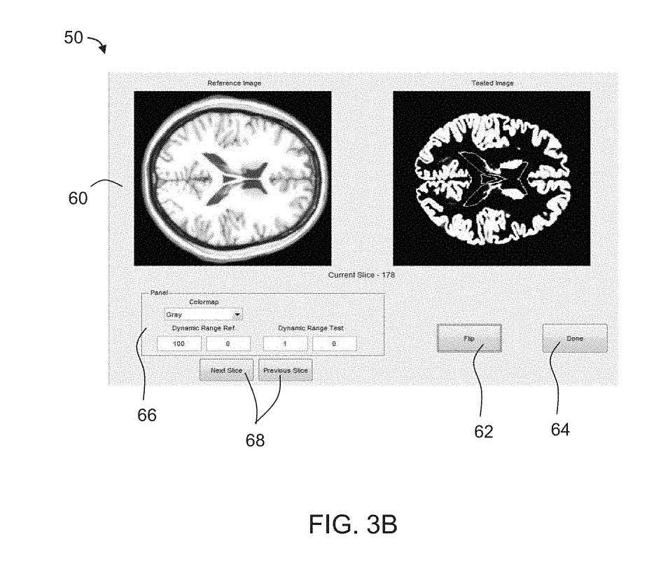

In some embodiments of the present invention the method displays 38 a graphical user interface (GUI) on a display device. The GUI can be displayed before execution of any operation of the method. Preferably, the GUI is displayed before obtaining the image and is updated during the constructions of the head model. A representative example of a GUI 50 suitable for the present embodiments is illustrated in FIGS. 3A and 3B. GUI 50 typically includes a head model viewing region 52 showing the constructed the head model. Optionally, GUI 50 also comprises an image viewing region 56 at which the input image is displayed.

GUI 50 can also comprise one or more sets of user input controls 54 displaying changeable parameters characterizing the head model. Three sets input controls 54 are shown in FIG. 3A, but GUI 50 can comprise less than three or more than three input controls, if desired. Each input control displays one or more changeable parameters for a different stage of the head model construction. For example, one set of input controls can allow the operator to select a 3D image of the head out of several possible images, one set of input controls can allow the operator to select parameters for the segmentation and smoothing of the inner part of the head, and one set of input controls can allow the operator to select parameters for the 3D reconstruction.

GUI 50 can also comprise one or more calculation activation controls 58. In the representative illustration of FIGS. 3A-B which is not to be considered as limiting, GUI 50 has a calculation activation control 58 for each set of input controls 54. Responsively to an activation of control 58 and to a change in the parameters in the respective set of input controls, the method preferably repeats the construction of head model using the new parameters. Optionally, GUI 50 allows the user to save the results obtained for a given stage using a given set of parameter, and to load previously saved results obtained for one stage as a basis for the execution of another stage.

FIG. 3B shows an optional window 60 of GUI 50 that displays the MRI scan, preferably after MNI conversion (FIG. 3B, left), and the same scan after segmentation (FIG. 3B, right). Window 60 is preferably displayed in response to an activation of one of controls 38. Window 60 can also include additional activation controls 62 and 64 that allow flipping and saving the segmented scan, respectively. Window 60 can also include an additional user input controls 66 displaying changeable viewing parameters (e.g., color, grayscale) for the MRI and the segmented scans, and slice selection controls 68 that allow the user to select the displayed slice of the for the MRI and the segmented scans.

The method ends at 39.

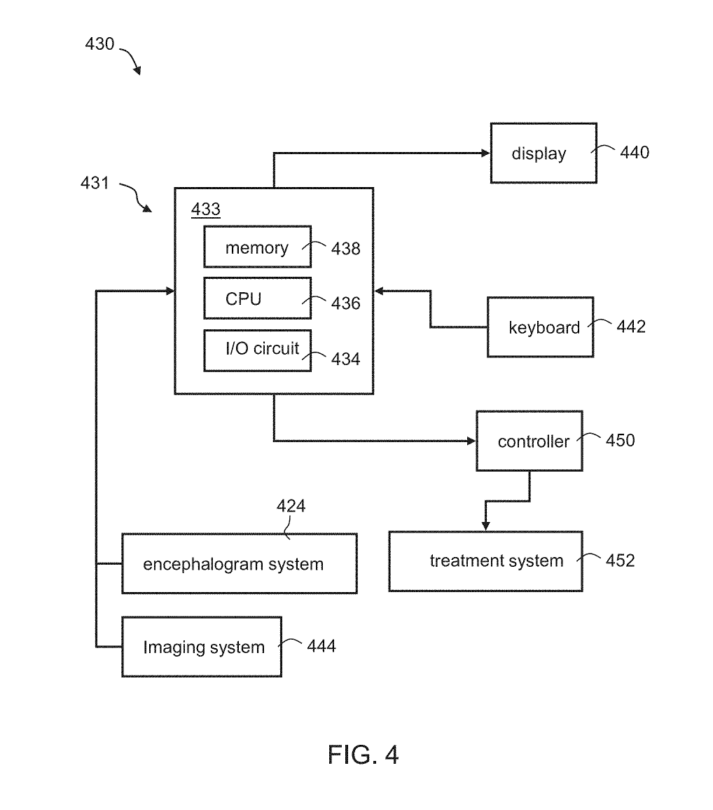

FIG. 4 is a schematic illustration of a system 430 suitable for estimating potential distribution over a cortical surface of a brain of a subject, and optionally also for treating a subject, according to some embodiments of the present invention. System 430 typically comprises a data processing system 431, which can comprise a computer 433, which typically comprises an input/output (I/O) circuit 434, a data processor, such as a central processing unit (CPU) 436 (e.g., a microprocessor), and a memory 446 which typically includes both volatile memory and non-volatile memory. I/O circuit 434 is used to communicate information in appropriately structured form to and from other CPU 436 and other devices or networks external to system 430. CPU 436 is in communication with I/O circuit 434 and memory 438. These elements are those typically found in most general purpose computers and are known per se.

A display device 440 is shown in communication with computer 433, typically via I/O circuit 434. Computer 433 issues to display device 440 graphical and/or textual output images generated by CPU 436. A keyboard 442 is also shown in communication with computer 433, typically I/O circuit 434.

It will be appreciated by one of ordinary skill in the art that system 431 can be part of a larger system. For example, system 431 can also be in communication with a network, such as connected to a local area network (LAN), the Internet or a cloud computing resource of a cloud computing facility.

Data processing system 431 is preferably configured for estimating potential distribution over a cortical surface of a brain of a subject having a head, for example, by executing method 10 and optionally also method 30.

In some optional embodiments of the present invention, system 430 comprises or is in communication with an EG system 424 (e.g., an EEG system, an MEG system or a combined EEG-MEG system) configured for sensing and/or recording the EG data and feeding data processor 433 with the data. In some optional embodiments of the present invention, system 430 comprises or is in communication with an imaging system 444 configured for imaging the head of the subject and feeding data processor 433 with the image. Preferably the image is a 3D image or a sliced image, as further detailed hereinabove.

In some optional embodiments of the present invention system 430 comprises a controller 450 configured for controlling a treatment system 452 (for example, a brain stimulation tool, hyperbaric therapy system, phototherapy tool) to apply treatment at parameters selected responsively to the result of the estimation of the cortical potential distribution, as further detailed hereinabove. In some embodiments of the present invention EG system 424, imaging system 444, processor 433 and controller 450 operate in a closed loop, wherein processor 433 estimates the cortical potential distribution, based on the data from systems 424 and 444, and wherein controller 450 adjusts the parameters of the treatment system 452 responsively to the estimation. Such a closed loop can be used to effect many types of changes in brain function. Representative examples include, without limitation, inducing of local neuroplasticity, inhibition of local activity, synchronization among different brain regions (nearby or remote from each other), and the like.

As used herein the term “about” refers to ±10%.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Example 1

In the present example, Laplace equation is solved in the bounded volume above the cortex and below the scalp using two boundary conditions: Scalp potentials acquired from measured EEG electrodes and cortex normal current distribution, estimated using a Surface Laplacian. The solution was constructed by finite element method (FEM) that takes into account tissue geometry and conductivity values acquired using MRI scans.

### Methods

Let the domain Ω consist of the volume between the scalp and cortex surfaces, ∂Ωₛ and ∂Ω_{c}, respectively. The Laplace Equation formulation can be written in the form:

$$\begin{matrix}∇ \cdot ( σ ∇ u ) = 0 r ∈ Ω & ( EQ . 1.1 ) \\ \frac{\partial u}{\partial n} = 0 r ∈ \partial {Ω}_{s} & ( EQ . 1.2 )\end{matrix}$$

where σ(r) is the (typically inhomogeneous) conductivity within the volume Ω and (EQ. 1.2) means that no normal current exits the scalp surface.

In the forward problem, the Laplace Equation is solved while the potentials on the cortical surface are assumed to be known and the potentials on the scalp surface are computed utilizing two boundary condition on both scalp and cortex surfaces. In the present example, however, a solution to the backward problem is described. In this solution, the Laplace Equation is solved while the potentials on the scalp surface are known from EEG measurement but are known on the cortex.

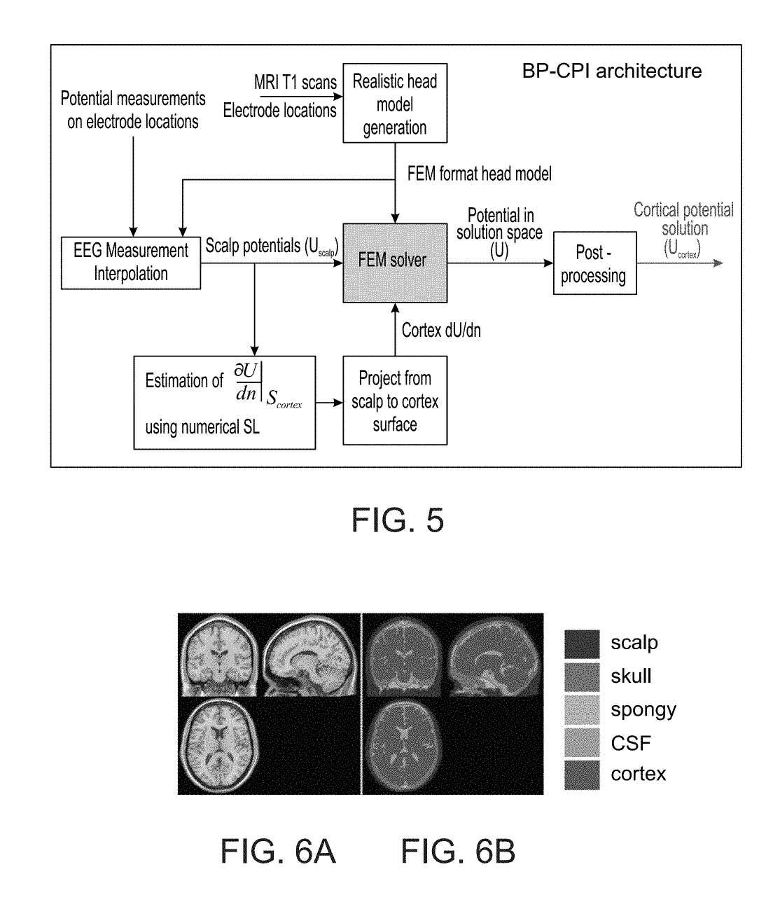

In the present Example, the following boundary conditions are employed:

$$\begin{matrix}\frac{\partial u}{\partial n} = g r ∈ {Ω}_{c} & ( EQ . 1.3 ) \\ u = p r ∈ \partial {Ω}_{s} & ( EQ . 1.4 )\end{matrix}$$

where p is the interpolated potential distribution acquired from the measured EEG electrodes and g is the surface Laplacian calculated on the scalp surface and projected onto the cortical surface. The process employed in the present example is illustrated in FIG. 5.

MRI Modeling

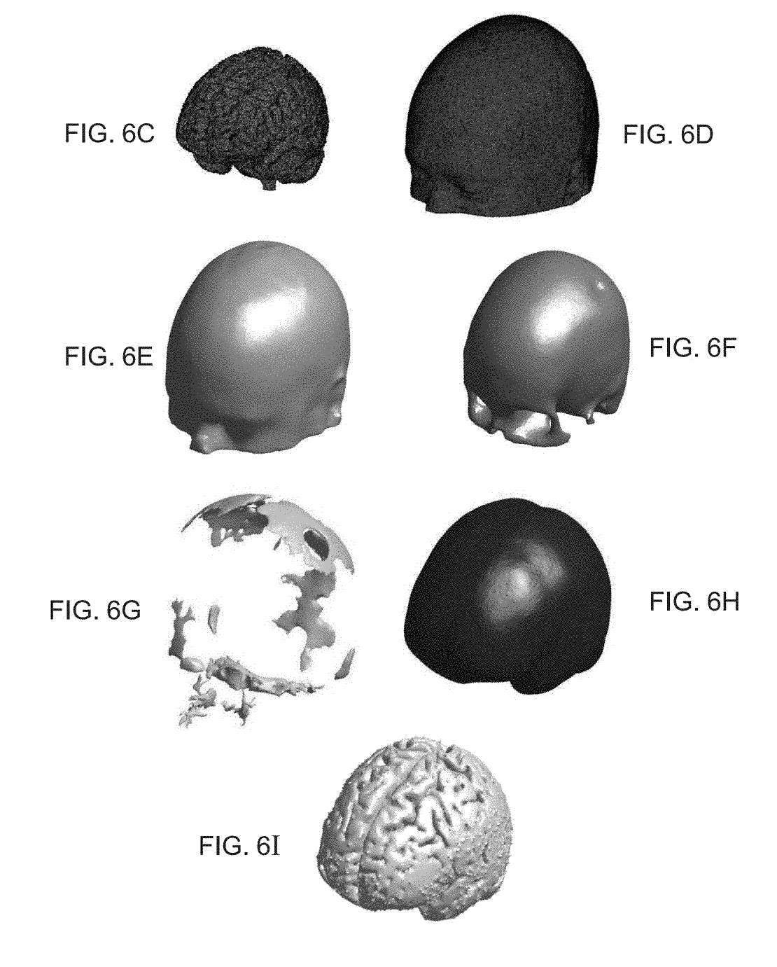

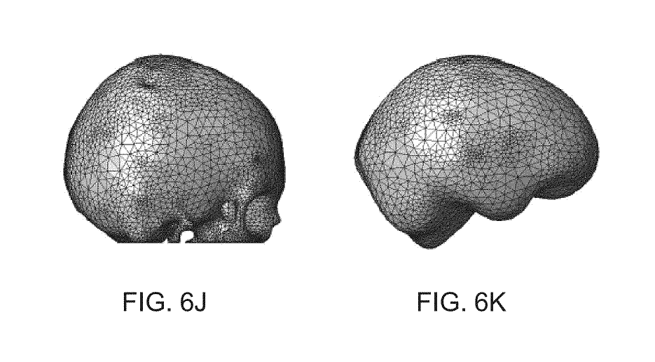

Semi-automatic segmentation and meshing algorithm was developed and included in the present study for constructing a head model. The constructed head model was a tetrahedral head mesh, partitioned into different sub-division for different tissues. In the present example, the iso2Mesh Matlab toolbox was employed for the 3D reconstruction. Each tissue received a homogenous isotropic conductivity value. The following tissues were defined for the head model: scalp, higher skull (compact bone), intermediate skull (spongy bone) and lower skull (compact bone) with conductivities 0.33, 0.0042, 0.0286 and 0.0042 S/m, respectively. The head model contained about 600,000 elements, 10,744 nodes on the scalp surface and 5,910 nodes on the lower skull surface. In this head model, the lower skull was selected to be the back-projected surface instead of the cortex surface and lower segmented tissues like the cerebral spinal fluid (CSF) and the cortex were ignored. FIGS. 6A-K show the obtained head model.

The Surface Laplacian

The Surface Laplacian operator is used in the present Example as an estimator for the normal currents flowing from the skull to the scalp layer. In the present Example, the method described in [J. Le, and A. Gevins, “Local estimate of surface Laplacian derivation on a realistically shaped scalp surface and its performance on noisy data”, Electroencephalography and clinical Neurophysiology. vol. 92, pp. 433-441. 1994] is used for calculating the Surface Laplacian. For the purpose of imposing cortical current as boundary condition, the Surface Laplacian results were projected onto the cortical surface. The projection was using the method described in [O. Masako and D. Ippeita, “Automated cortical projection of head-surface locations for transcranial functional brain mapping”, NeuroImage. vol. 26 pp. 18-28.2005].

Finite Element Method

The variational principle for the Laplace Equation provides the functional F(u), where u is the potential function in Ω. Its minimum is equal to the solution of the Laplace Equation.

$$F ( u ) = \frac{1}{2} {\int \int \int }_{V} [ - ∇ ( σ ∇ u ) ] \cdot u * \cdot dv \qquad ( EQ . 1.5 )$$

Using Green's theorem F can be written as:

$$F ( u ) = \frac{1}{2} {\int \int \int }_{V} σ {\vert ∇ u \vert}^{2} \cdot dv - \frac{1}{2} {∯}_{{Ω}_{s} + {Ω}_{c}} σ u \frac{\partial u}{\partial n} ds \qquad ( EQ . 1.6 )$$

The surface integral can be simplified by using the fact that no current exits the head, and introducing the estimation to the cortical normal derivative from (EQ. 1.3):

$$\frac{\partial u}{\partial n} = 0 r ∈ {Ω}_{s} \qquad ( EQ . 1.7 )$$

FEM formulation of the volume integral can be done using tetrahedron elements of the first order, which results in the functional matrix representation:

$$F ( u ) = \frac{1}{2} {\sum }_{e = 1}^{{M}_{e}} {[ {\underline{u}}^{e} ]}^{T} \cdot [ {\underline{\underline{S}}}^{e} ] \cdot [ {\underline{u}}^{e} ] - \frac{1}{2} {\sum }_{s = 1}^{{M}_{s}} {[ {\underline{q}}^{s} ]}^{T} \cdot [ {\underline{\underline{K}}}^{s} ] \cdot [ {\underline{u}}^{s} ] \qquad ( EQ . 1.8 )$$

where Mₑ is the number of elements, [u^{e}] denotes the vector of potentials at nodes in the element e and [S^{e}] is the element kernel matrix which is evaluated using linear basis functions Nᵢ^{e}(x,y,z):

$${[ {\underline{\underline{S}}}^{e} ]}_{i , j} = {\int \int \int }_{V} σ ( x , y , z ) [ \frac{\partial {N}_{i}^{e}}{\partial x} \cdot \frac{\partial {N}_{j}^{e}}{\partial x} + \frac{\partial {N}_{i}^{e}}{\partial y} \cdot \frac{\partial {N}_{j}^{e}}{\partial y} + \frac{\partial {N}_{i}^{e}}{\partial z} \cdot \frac{\partial {N}_{j}^{e}}{\partial z} ] \cdot dv \qquad ( EQ . 1.9 )$$

[q^{s}] is the vector containing the values of the estimated normal derivative at the cortical nodes and:

$${⌊ {\underline{\underline{K}}}^{s} ⌋}_{ij} = {σ}_{s} {\int \int }_{s} {N}_{i}^{s} \cdot {N}_{j} \partial s \qquad ( EQ . 1.10 )$$

Taking the first derivative of (EQ. 1.8) and equating the result to 0 we can build the next matrix equations to find the potentials inside the solution volume:

$$\begin{matrix} ⌊ {\underline{\underline{A}}}_{g} ⌋ \cdot ⌊ {\underline{u}}_{g} ⌋ = ⌊ {\underline{b}}_{g} ⌋ & ( EQ . 1.11 ) \\ where & \\ [ {\underline{\underline{A}}}_{g} ] = {\sum }_{e = 1}^{{M}_{e}} [ {\underline{\underline{S}}}^{e} ] [ {\underline{u}}_{g} ] = {\sum }_{e = 1}^{{M}_{e}} [ {\underline{u}}^{e} ] [ {\underline{b}}_{g} ] = - \frac{1}{2} {\sum }_{s = 1}^{{M}_{s}} {[ {\underline{q}}^{s} ]}^{T} \cdot [ {\underline{\underline{K}}}^{s} ] & ( EQ . 1.12 )\end{matrix}$$

Next, two internal boundary condition are imposed upon our solution through linear constraints on (EQ. 1.12). The first is expressed in (EQ. 1.3) and the second is the imposition of a continual normal current on every surface Sᵢ bounded by two different tissues, as shown in (EQ. 1.13):

[σ₂∇u⁺−σ₁∇u⁻]·\hat{n}|ₛ_{ₛ}=0 (EQ. 1.13)

which can be shown to yield the next relation:

u^{s}=α·u⁻+(1−α)·u⁺ (EQ. 1.14)

where u⁺ and u⁻ are the potentials at a point located Δn⁺ above and Δn⁻ below a specific vertex u^{s} on Sᵢ, respectively, and a is the constant relating the tissues conductivity ratio χ₂₁ according to (EQ. 1.15).

α=[1+χ₂₁(Δn⁻/Δn⁺)]⁻¹ (EQ. 1.15)

Both internal boundary conditions are enforced in the present example through evaluating the vector [c] and matrix └P┘ that impose linear constraints on the └u_{g}┘ by the relation:

└u_{g}┘=[c]+└P┘·└u_{g}┘ (EQ. 1.16)

where [c] is the vector imposing direct potential values (Dirichlet's boundary condition) and └P┘ is a matrix dictating linear dependences as shown in (EQ. 1.14). Once done, (EQ. 1.11) and (EQ. 1.12) take a different form as shown in (EQ. 1.17) and (EQ. 1.18) below, to find the final potential distribution within the solution volume:

└A_{g}′┘·[u_{g}]−└b_{g}′┘ (EQ. 1.17)

└A_{g}′┘=[P]^{T}·└A_{g}┘·[P] [b_{g}′]=[P]^{T}·*└b_{g}┘−└A_{g}·[c]) (EQ. 1.18)

### Results

A validation process was implemented in two stages.

Validation on Concentric Spheres Head Model

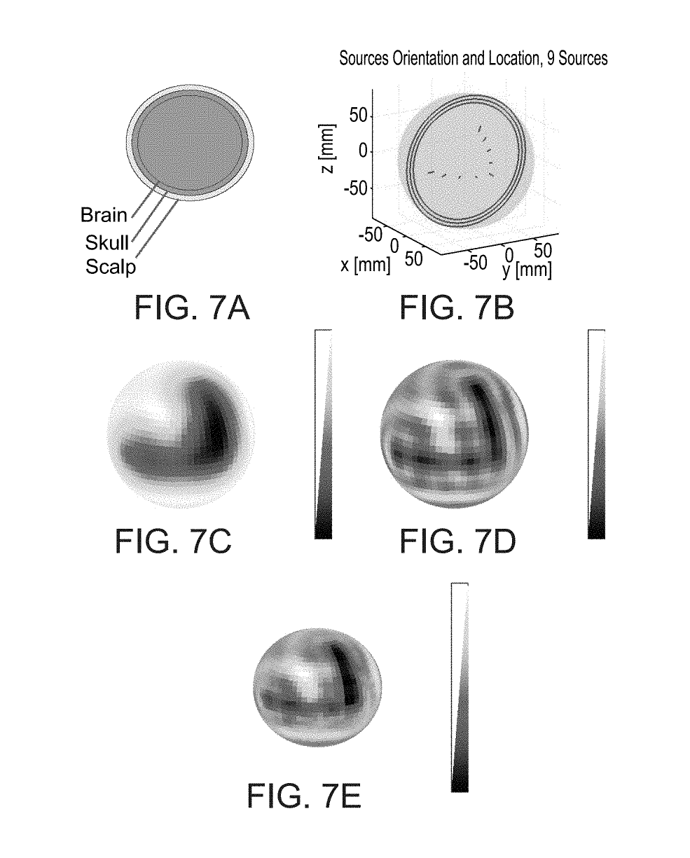

The first validation stage was to apply the method described in this Example on scalp potential distribution for a Three Concentric Sphere Head Model (TCSHM). This head model was composed of three concentric spheres that model the scalp, skull and cortex tissues, as seen in FIG. 7A. Each tissue was assigned with conductivity value extracted from the literature and their radii was selected to be 9, 8.5 and 8 cm, respectively. The analytical potential distribution on the scalp and cortex surfaces was calculated for the case of nine, L shaped, normal dipole structure as shown in FIG. 7B. Then the method described in the present Example was applied on the scalp potential distribution. The analytical solution of the scalp and cortex potential distribution is shown in FIGS. 7C-D and the estimated cortical potential distribution is shown in FIG. 7E. A comparison between the surface Laplacian as calculated analytically and numerically is provided in Table 1, below.

TABLE 1

| | | Numerical | |
|---|---|---|---|
| | Analytical Calculation | Calculation | Real dU/dn |
| Analytic SL | 1 | 0.996 | 0.993 |
| Numeric SL | 0.996 | 1 | 0.994 |
| Real dU/dn | 0.993 | 0.994 | 1 |

The technique of the present embodiments provides a good estimation for the cortical potential, and the calculated Pearson's correlation coefficient (CC) between the analytical and estimated cortical potentials was very high (CC=0.996).

Validation on Realistic Head Model

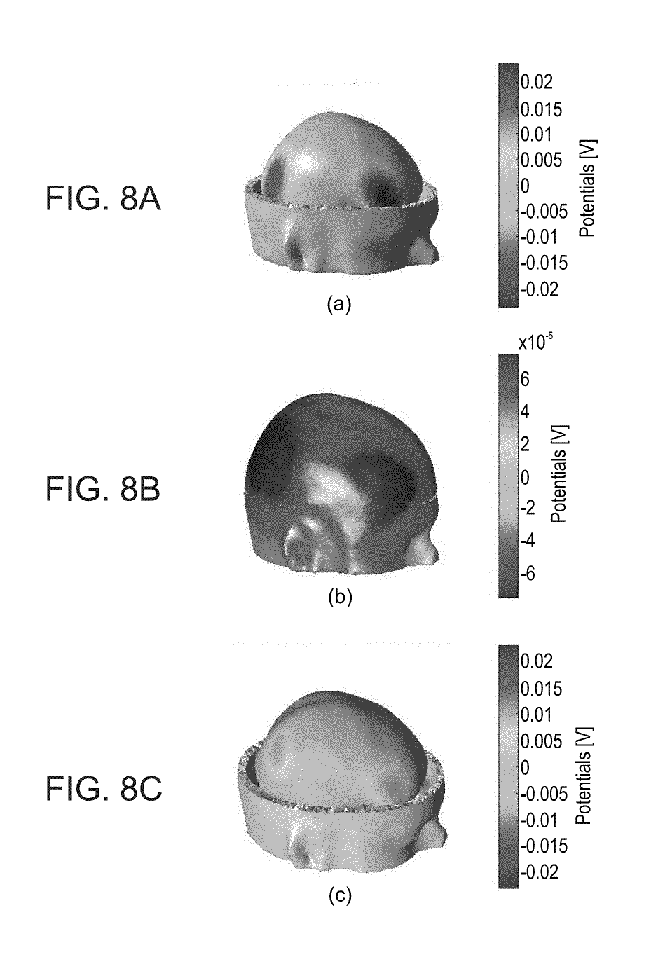

In the realistic head model the scalp, compact boned skull, spongy boned skull and the lower skull surfaces and assigned conductivity values. In this example, conductivity values for the CSF and cortical surface were not assigned. The estimation is projected to the lower skull (Lskull) surface and is treated as the cortical potentials. In is postulated that the potential distribution over the Lskull and over the cortex surface are not significantly different because of the relatively high conductivity values in the volume below the Lskull surface. In this validation stage a Forward-Backward validation technique was employed. Starting with an initial pre-defined Lskull potential distribution as the excitation, the scalp potential distribution due to the excitation was calculated (forward solution). Then, the method described in this example was executed only knowing the scalp potentials to find the cortical potential estimation (backward solution). The backward solution was to compare the initial excitation potential. The results are shown in FIGS. 8A-C. As shown there is a good agreement (CC=0.69) between the initial and estimated distributions cortical distributions.

### Example 2

EEG typically records electrical activities arising from sites other than the brain. The recorded activity that is not of cerebral origin is termed artifact and can be originated in physiologic (e.g., ocular, muscles, glossokinetic and ECG activities) and extraphysiologic artifacts (e.g., interferences, such as, but not limited to, electrode movement and equipment electrical interferences. In addition, measurement errors e.g., insufficient scalp electrodes, electrode displacements also influence on EEG. Artifacts and measurement errors can cause misinterpretation of EEG signals, and affect cortical potential estimation by CPI techniques.

The above artifacts, interferences and errors can be overcome by using a technique known as the event-related potentials (ERP). In this technique an average measure of the brain response is calculated as a direct result of a specific sensory, cognitive, or motor event or stimuli. Due to this averaging procedure only synchronized (to the stimuli) responses are accumulated, and all other signals that are not originated in brain electrical activity, are diminished. Even though many of the mentioned undesired signals can be reduced, some cannot be eliminated, and are optionally and preferably accounted for when reviewing a high-resolution EEG technique. Considering the effect of such undesired signals on the CPI provides better interpretation of the results.

In the present Example, a practical simulative validation is performed to investigate and characterize the sensitivity of the back-projection CPI (BP-CPI) procedure to different types of realistic interferences and errors.

Six types of parameter modulations were used to examine the procedure's competence to estimate the cortical potentials under different electrode noise level, various number of scalp electrodes, changes in electrode displacement on the scalp, errors in head tissue conductivity estimation, depth and spread of the sources inside the brain volume.

### Materials and Methods

Validation Design

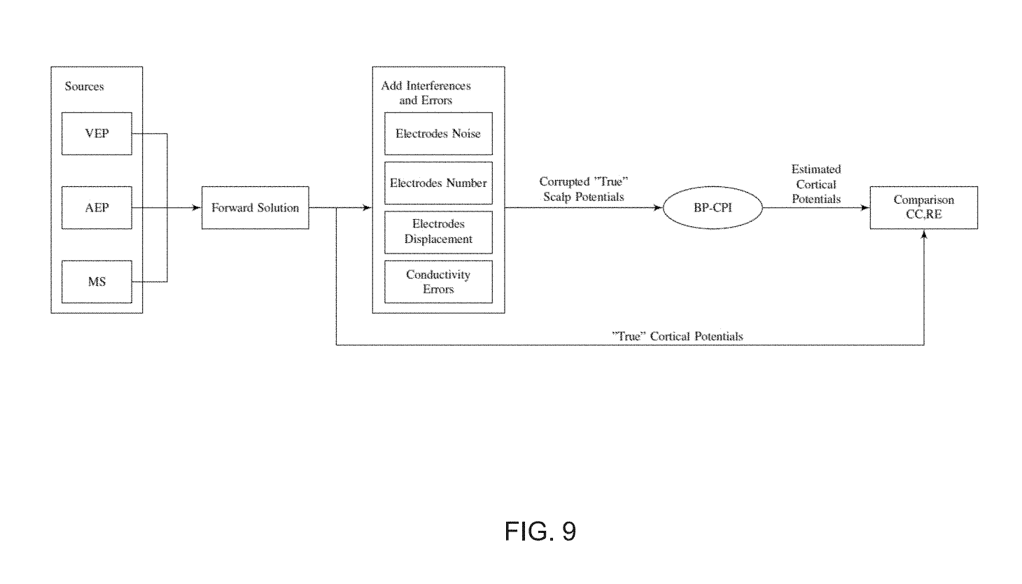

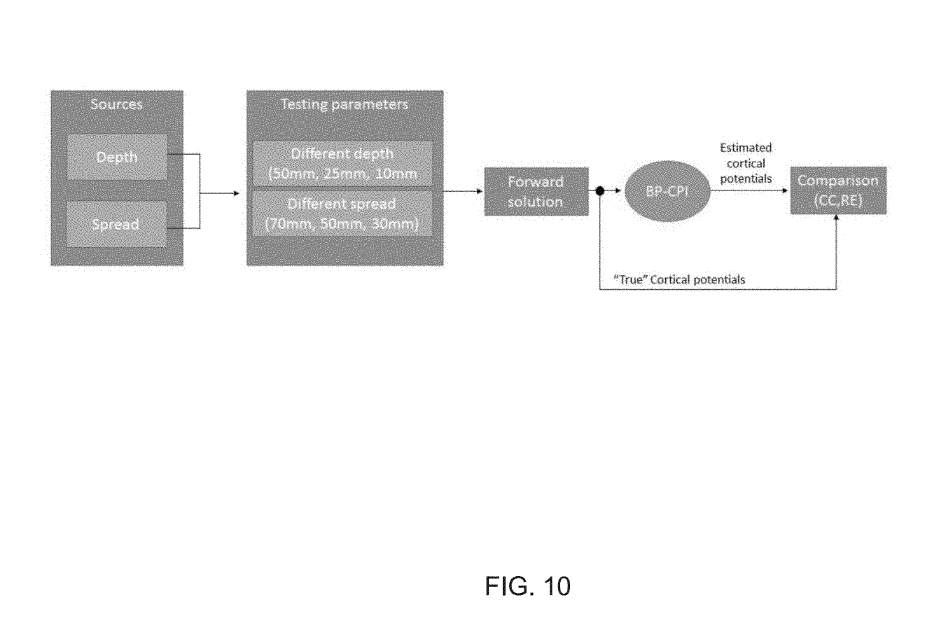

The validation design process was split into two validation channels. A first channel examines real-life interferences and errors effects on BP-CPI, as presented in FIG. 9. Three types of sources distributions were used: visual evoked potential (VEP), auditory evoked potential (AEP) and manually selected (MS). Each test included one distribution. The selected distribution was used to maintain the “true” scalp and cortical potentials (the forward solution) by importing the sources locations, orientation and strength into an electromagnetic (EM) simulation software (Sim4Life 2.0 by ZMT), in addition to the realistic head model conductivity properties. Once “true” scalp potentials were found, different interferences and errors were added and the resulted modified potentials were used for the BP-CPI cortical potential estimation. Then, the “true” and estimated cortical potentials were compared using the Pearson's CC and the relative error (RE) measures. A second channel examined the BP-CPI performance in the presence of sources at different depths and spread, as depicted in FIG. 10. Similar scheme was used as in the first validation channel, but here, the BP-CPI scalp potentials were not corrupted and the BP-CPI was tested for different sources depths and spread.

It was found by the Inventor that the above two-channel procedure allowed examining the performance of BP-CPI under different realistic environment interferences, real-life error and sources depth and spread with small or no bias.

Head Modeling

The head modeling was based on a single T1 weighted MRI scan of a subject head, with an in-plane resolution of 0.67 mm by 0.67 mm and slice thickness of 1 mm (3-D MP-RAGE, TR=2000 ms, TE=2.99 ms, 8° flip). The MRI scan was aligned and normalized according to the MNI template ICBM152, to allow co-registration and comparison between subjects. An automatic algorithm was applied to segment relevant head tissues and bone. The different surfaces extracted by the realistic head modeling algorithm are shown in FIGS. 6E-6I. Shown are scalp layer (FIG. 6E), skull layer (FIG. 6F), diploe layer (FIG. 6G), lower skull (Lskull) layer (FIG. 6H) and cortex layer (FIG. 6I).

Meshing and assignment of conductivity properties for each layer was also performed by the automatic algorithm as described in Example 3, below. Electrodes for different systems were matched to the extracted scalp surface using a multidimensional optimization. The estimation was projected to the Lskull surface which is referred to as the cortical surface with cortical potentials. It is assumed that the potential distribution over the Lskull and the cortex surfaces are similar, due to relatively high conductivity values in the volume below the Lskull surface and closeness of the two surfaces.

Electrodes Systems

Four EEG electrodes systems were used in the tests. All the systems were based on commercial EEG systems. The electrodes locations were extracted from manufacturer data-sheets, which are given in MNI coordinates, as is the realistic head models. These MNI locations were given for an unknown head normalized to MNI space. The electrodes systems that were employed include the EGI 128 net (by Electrical Geodesic. Inc.) including 128 electrodes, the EasyCap 64 net (by Brain Products) including 64 electrodes based on the 10-5 universal system, a 29 electrode system based on the 10-5 universal system, and a 9 electrode system based on the 10-5 universal system. For the EGI 128 electrodes net and the EasyCap 64 electrodes net, electrodes below the ear line were removed. This procedure discarded 4 electrodes from the EGI 128 net and 2 electrodes from the EasyCap 64 net, leaving a 124 electrode system and a 62 electrode system, respectively.

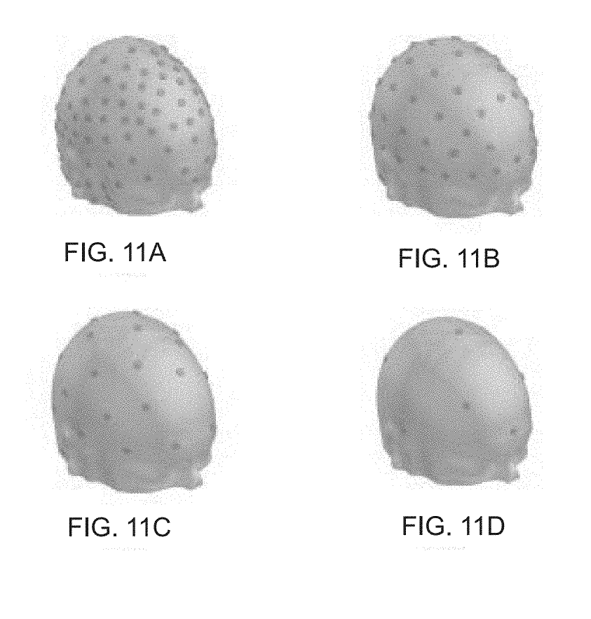

To adjust the location to the head model, an iterative optimization scheme was used to find the best linear transformation for the respective location to the head model. This transformation was used on all realistic head models. FIGS. 11A-D illustrate the electrodes systems that were used, shown are a 124 electrode system (FIG. 11A), a 62 electrode system (FIG. 11B), a 29 electrode system (FIG. 11C) and a 9 electrode system (FIG. 11D).

Reference Simulated Data

For the process of validation, three reference simulated data was generated. A single or multiple dipole sources with different orientations were used to represent a single or multiple well-localized areas of brain electric activity. Two of these distributions are based on sources locations for visual and auditory evoked potential, respectively [Di Russo et al., 2002, Human brain mapping, 15, 2, pages 95-111; Huotilainen et al., 1998, Electroencephalography and Clinical Neurophysiology/Evoked Potentials Section, 108, 4, pages 370-379]. An additional manually selected source distribution was used to test the BP-CPI in more cortical areas not tested in the VEP and AEP.

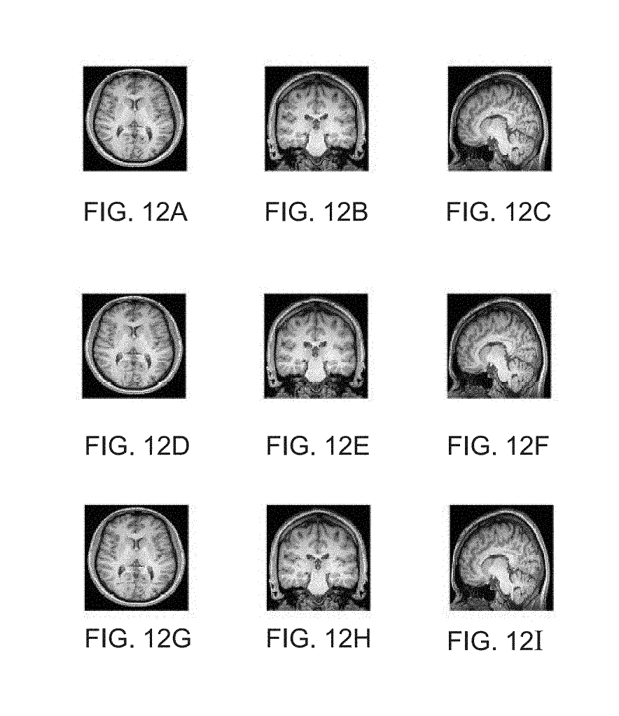

FIGS. 12A-I show sources distributions for the BP-CPI validation on realistic head model. FIGS. 12A, 12D and 12G show axial views, FIGS. 12B, 12E and 12H show coronal views, and FIGS. 12C, 12F and 12I show sagittal views of the subject MRI. Shown are validations for two AEP source locations and orientations (FIGS. 12A-C), seven VEP source locations and orientations (FIGS. 12D-F), and five MS sources locations and orientations (FIGS. 12G-12I). The source distributions are shown in FIGS. 12A-I as red arrows marking the location and orientation of the sources.

Simultaneous EEG and fMRI Measurements

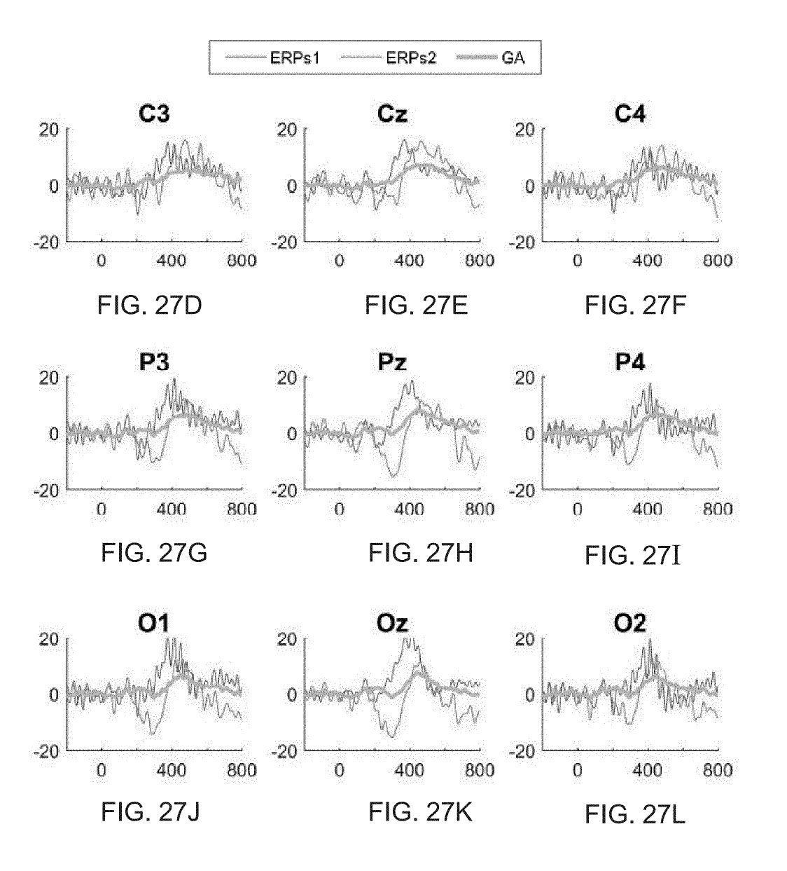

Thirty eight healthy subjects participated in a simultaneous recording of EEG and fMRI. The visual Go-No-go task was selected for the purpose of validating the BP-CPI performance in comparison to fMRI activations. In this task, a frequent “Go” stimulus, which, in this Example, occupied 80% of all trials, required the subject to perform a motor response each time it appears on the screen. A rare “No-go” stimulus (in this Example, 20% of all trials) required the subject to refrain from responding. The Go stimuli in this Example consisted of white English alphabetic letters (B, C, D, etc.), appearing in equal proportions, and the No-go stimulus was a white X symbol. The subjects viewed the stimuli through a mirror that was placed on the upper part of the head coil. Four blocks of 90 stimuli each were presented and the duration of the task was approximately 12 min. A 10 trial practice block was run prior to the experimental session. MRI data was collected during the experiment, and the same averaged MRI scan was used to generate the realistic head model for the BP-CPI estimation. Scalp potentials were recorded using the 62 electrode system described above. Gradient and ballistocardiogram artifact removal procedure was performed on the resulted EEG raw data. Then, ERP was generated for each subject which was averaged to maintain a grand average for each of the stimuli. For the validation process, ERP were extracted from two specific subjects (s1 and s2). These ERPs are denoted in this Example by ERPs1 and ERPs2. The grand average, ERPs1, and ERPs2 (no-go stimuli) for selected electrodes are presented in FIGS. 27A-L. In FIG. 27A-L the vertical axis is amplitude in units of tV, the horizontal x axis is time in units of ms.

### Results

Every test was followed by visual inspection for one of the simulated source distribution and quantitative evaluation for all distributions.

Noise of different power was added to the electrodes potentials acquired from the reference simulated data to generate scalp potentials of various signal to noise ratio (SNR), calculated as follows:

SNR=max{ϕ_{scalp}}/σ_{elec}

where σ_{elec} is the standard deviation of the additive white Gaussian noise added to the noise free scalp electrodes potentials ϕ_{scalp}.

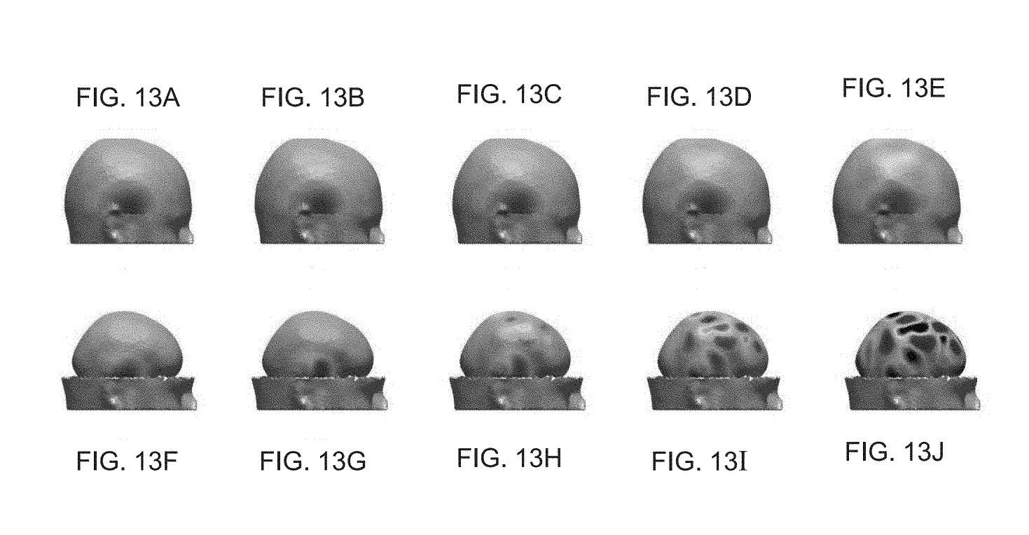

FIGS. 13A-J depict results of true scalp and cortical potentials associated to the AEP sources, along with BP-CPI results, while adding noise with different power. Shown are scalp (FIGS. 13A-E) and cortical (FIGS. 13F-J) potentials, for a reference solution (FIGS. 13A and 13F), 0% noise (SNR=∞) (FIGS. 13B and 13G), 2.5% noise (SNR=40) (FIGS. 13C and 13H), 5% noise (SNR=20) (FIGS. 13D and 13I) and 10% noise (SNR=10) (FIGS. 13E and 13J). Good localization of sources is maintained even though high levels of noise is added which give additional artifacts to the cortical spatial pattern.

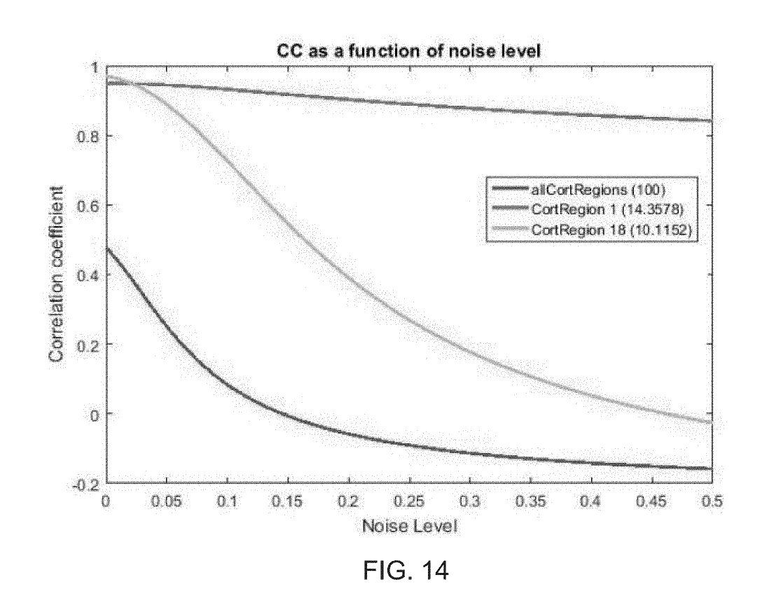

FIG. 14 shows quantitative measure of the estimation quality as a function of noise level. A monotonic decrease in CC is shown due to lower SNR. Additionally, relevant area of high amplitude provides high resemblance (CC>0.8) for SNR above 10.

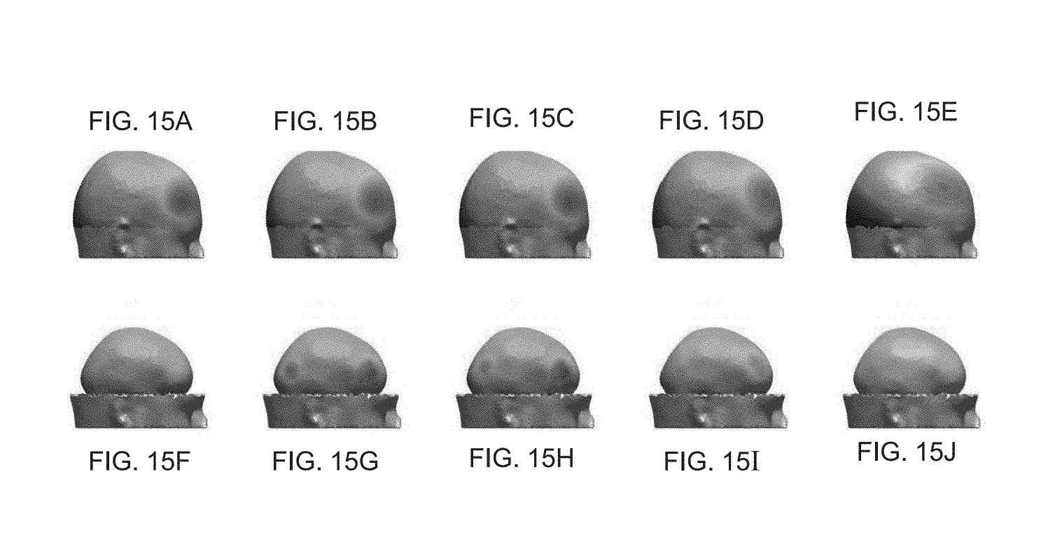

FIGS. 15A-J show the scalp potentials sampled with different sets of electrodes, and the corresponding estimated cortical potentials. Shown are scalp (FIGS. 15A-E) and Lskull (FIGS. 15F-J) potentials, for a reference solution (FIGS. 15A and 15F), and four electrode systems: EGI124 (FIGS. 15B and 15G), BP62 (FIGS. 15C and 15H), BP29 (FIGS. 15D and 15I) and BP9 (10-20) (FIGS. 15E and 15J). As shown, the BP-CPI solutions with 124 (FIGS. 15B and 15G) and 62 (FIGS. 15C and 15H) electrodes are much more accurate than the solution obtained with other electrodes sets which underestimates the potentials at the cortical surface, especially for occipital sources. Table 2, below, provides quantitative measures (Pearson's CC and RE), demonstrating same trend for all other cortical activations.

TABLE 2

| | | No. of electrodes | | | | | | | |
|---|---|---|---|---|---|---|---|---|---|
| | Region | 124 | | 62 | | 29 | | 9 | |
| Sources | of measurement | CC | RE | CC | RE | CC | RE | CC | RE |
| VEP | All cortex | 0.78 | 0.1 | 0.77 | 0.14 | 0.71 | 0.33 | 0.65 | 0.76 |
| | ROI | 0.97 | 0.04 | 0.91 | 0.08 | 0.88 | 0.21 | 0.67 | 0.42 |
| AEP | All cortex | 0.82 | 0.12 | 0.73 | 0.28 | 0.71 | 0.34 | 0.38 | 1.81 |
| | ROI | 0.98 | 0.05 | 0.93 | 0.09 | 0.9 | 0.1 | 0.21 | 0.87 |
| MS | All cortex | 0.75 | 0.41 | 0.74 | 0.54 | 0.56 | 0.98 | 0.25 | 2.23 |
| | ROI | 0.93 | 0.1 | 0.91 | 0.17 | 0.75 | 0.61 | 0.25 | 1.55 |

The electrode displacement error is defined as the distance between the actual location of the electrode on the subject's scalp, referred to in this example as the “real” location, and the location used by the BP-CPI process, referred to in this example as the “virtual” location. This type of error can be generated by electrode set misplacement by the EEG technician or by variation between the averaged locations obtained from the EEG electrodes manufacturer. In this sensitivity test, reference simulated scalp potentials were sampled in the “actual” locations, but interpreted by the BP-CPI process as in the “virtual” location. The displacement errors for each of the electrodes were selected randomly with normal distribution and changing standard deviation in order to test different levels of displacement error.

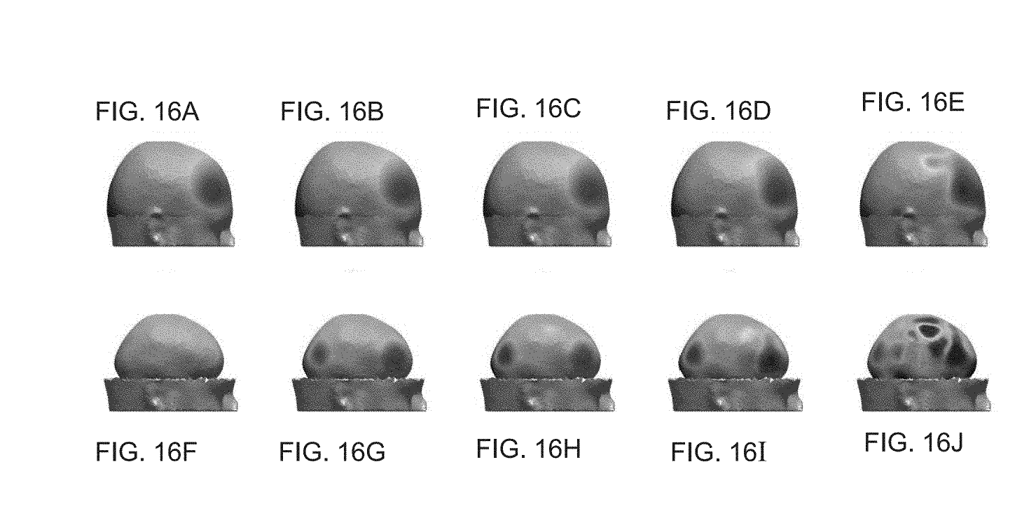

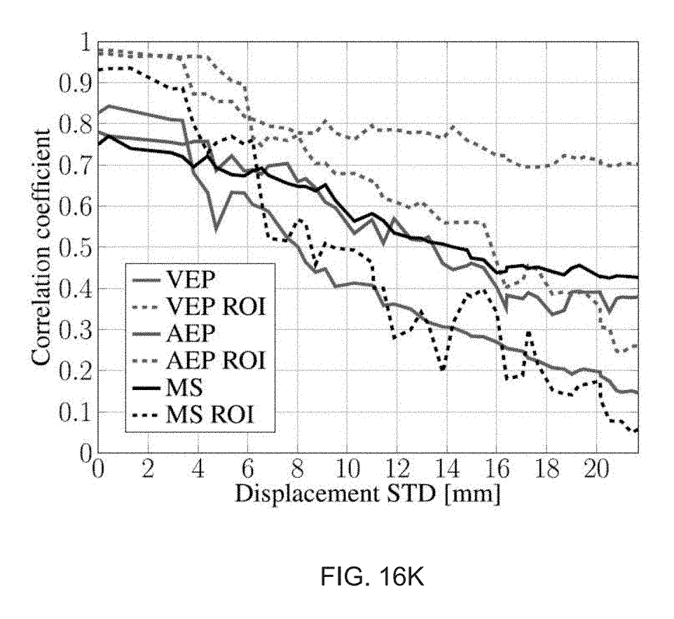

FIGS. 16A-J show electrode displacement error sensitivities for the scalp (FIGS. 16A-E) and Lskull (FIGS. 16F-J) potentials, for a reference solution (FIGS. 16A and 16F), and four values of the displacement noise: 0 mm (FIGS. 16B and 16G), about 2 mm (FIGS. 16C and 16H), about 4 mm (FIGS. 16D and 16I) and about 14 mm (FIGS. 16E and 16J). FIG. 16K is a graph showing the CC measures as a function of the displacement standard deviation for all source types. As shown in FIGS. 16A-K, for all types of sources, the BP-CPI of the present embodiments estimates the cortical potential with sufficient correlation on both the region-of-interest and the overall cortical surface, for errors distribution with standard deviation of less than 5 mm.

The compact bone part of the skull has a much lower conductivity than all other layers and in some embodiments of the present invention forms a part in the inventive BP-CPI process. The effect of local conductivity variations over the skull surface can also affect the EEG measured potentials. Variations in skull conductivity can arise from differences in measurement methods as well as from normal variations in skull conductivity between subjects due to differences in, for example, age, sex, illness, weight, and daily water consumption. The nominal conductivity of the boney skull used in this Example is 0.0125 as commonly used in neural source localization techniques [Rush et al., 1968, Anesthesia & Analgesia, 47, 6, pages 717-723].

For the purpose of measuring the BP-CPI sensitivity to skull conductivity variations the forward solution was solved five times with different skull conductivities between −40 and 40 percents from the nominal conductivity values, each time solving the BP-CPI when assuming skull conductivity as the nominal one.

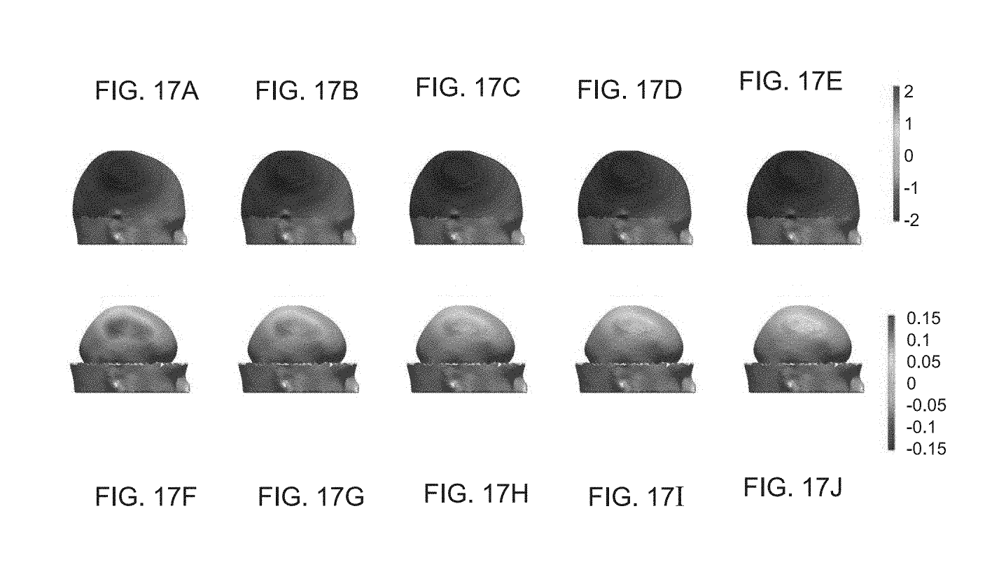

FIGS. 17A-J show conductivity estimation sensitivities for the scalp potentials (FIGS. 17A-E color scale in units of mV) and cortex potentials (FIGS. 17F-J color scale in units of V), using the HydroCel Geodesic EEG sensor net (Electrical Geodesic Inc.) with 128 electrodes from which with 4 electrode discarded from analysis, resulting in a 124 electrode system. Shown are results for +40% of the nominal conductivity value, corresponding to σₛₖᵤₗₗ= 1/32 S/m (FIGS. 17A and 17F), +20% of the nominal conductivity value, corresponding to σₛₖᵤₗₗ= 1/48 S/m (FIGS. 17B and 17G), the nominal conductivity value (σₛₖᵤₗₗ= 1/80 S/m) (FIGS. 17C and 17H), −20% of the nominal conductivity value, corresponding to σₛₖᵤₗₗ= 1/96 S/m (FIGS. 17D and 17I), and −40% of the nominal conductivity value, corresponding to σₛₖᵤₗₗ= 1/112 S/m (FIGS. 17E and 17J). For all cases, only skull conductivity was varied, and other tissues conductivity remain constant.

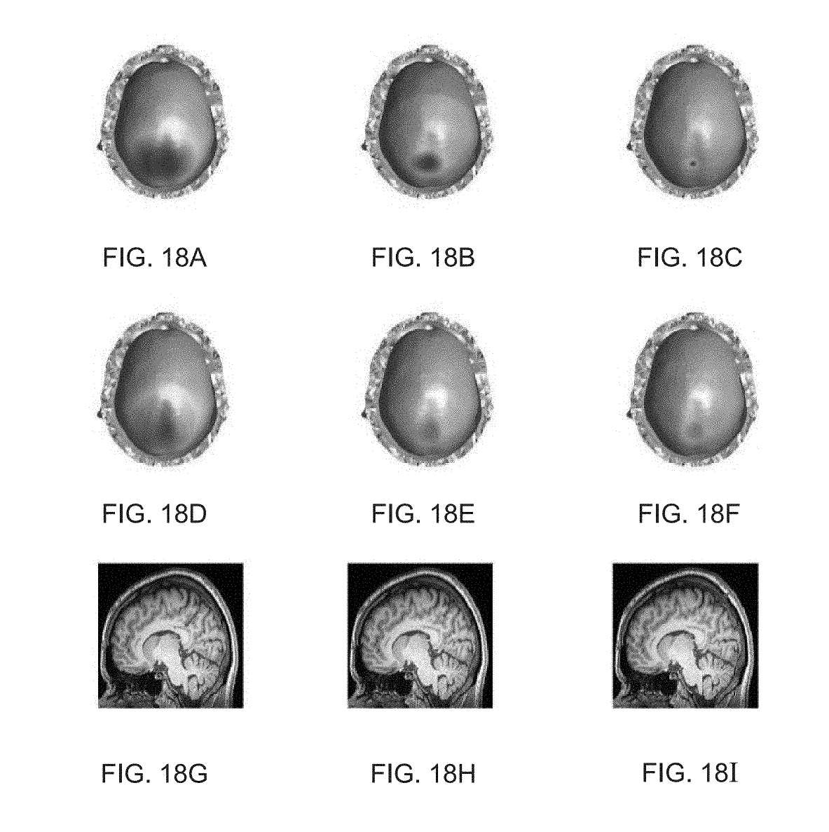

EEG recording are mostly the outcome of brain sources located at the higher part of the cortex. The deeper the source is located the higher the spread of potentials on the scalp. The sensitivity of the BP-CPI of the present embodiments to the depth of the source has been examined by the Inventors. One normally oriented source was placed normal to the parietal part of the cortex located at a depth of 10 mm, 25 mm and 50 mm. This cortical region was selected because it involves relatively non-smooth surface, and very thick skull region that results in high “blurring” effect. The results are presented in FIGS. 18A-I, where FIGS. 18A, 18D and 18G correspond to depth of about 50 mm, FIGS. 18B, 18E and 18H correspond to depth of about 25 mm, and FIGS. 18C, 18F and 18I correspond to depth of about 10 mm. FIGS. 18A-C show the reference cortical potentials, and FIGS. 18D-F show top views of the BP-CPI estimation where the potential values were quantized to 25 levels, sagittal views of the examined source locations and orientation are displayed in FIGS. 18G-I. FIGS. 18A-I demonstrate that there is a good localization of the source position, even though BP-CPI give wider representation of the cortical potentials generated by the source.

Experiments have also been conducted to evaluate the ability of the technique of the present embodiments to separate close sources. Four sources were placed in the center region of the cortex, about 10 mm beneath the cortical surface with a changing distance between them. This spread was selected to be 70 mm, 50 mm and 30 mm.

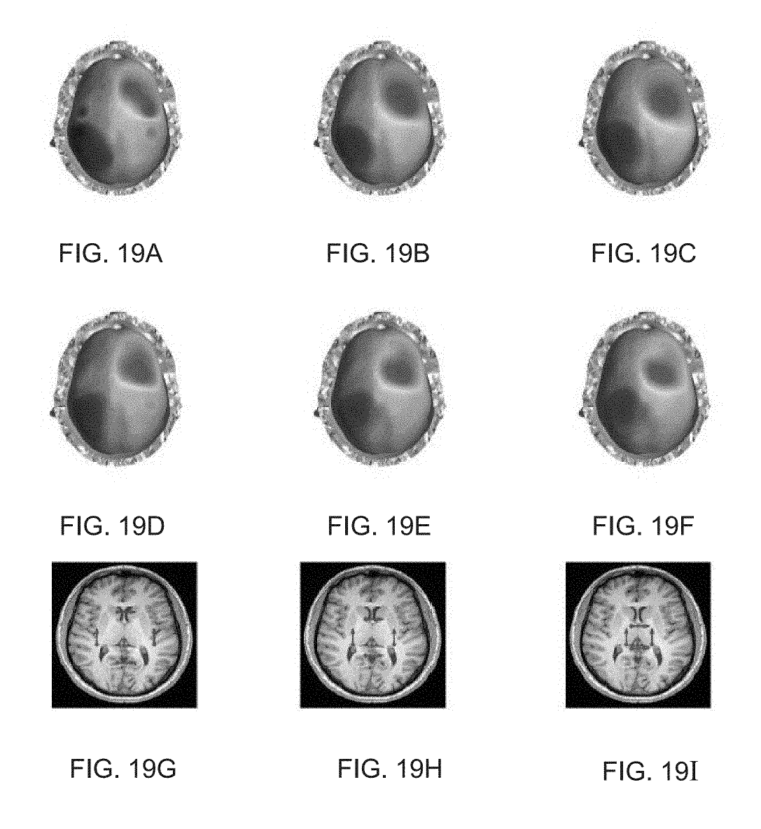

The results are presented in FIGS. 19A-I, where FIGS. 19A, 19D and 19G correspond to source spread of about 70 mm, FIGS. 19B, 19E and 19H correspond to source spread of about 50 mm, and FIGS. 19C, 19F and 19I correspond to source spread of about 30 mm. FIGS. 19A-C show the reference cortical potentials, and FIGS. 19D-F show top views of the BP-CPI estimation where the potential values were quantized to 25 levels. Sagittal views of the examined source locations and orientation are displayed in FIGS. 19G-I. FIGS. 19A-I present the case of tangential sources, but similar results were obtained with normal sources. FIGS. 19A-I demonstrate that there are good localization of the sources position and cortical potentials for all spreads, although for very close sources (a 30 mm in the present example) some artificial smearing of cortical potentials was developed.

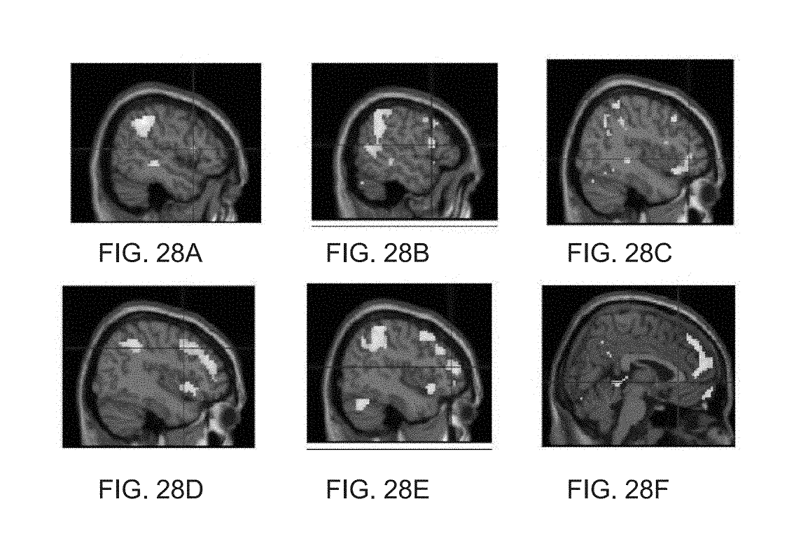

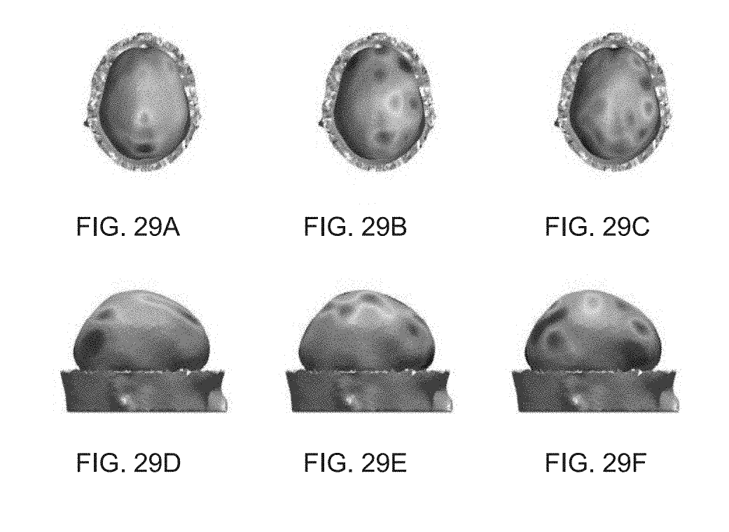

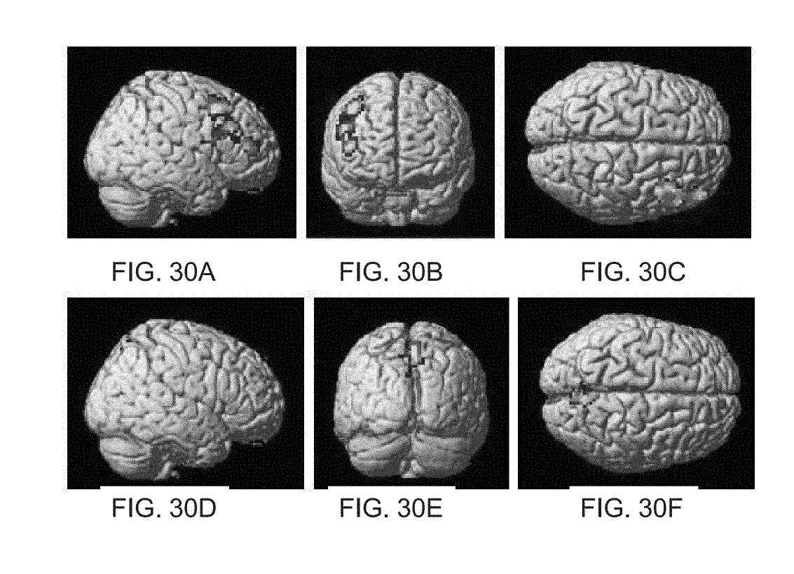

Cortical potential maps were generated using the BP-CPI technique of the present embodiments the grand average, and the ERPs of subjects s1 and s2. Group and single subject fMRI analysis was done to find activated brain areas for the no-go condition. In this Example, only cortical activations were addressed. The experimental validation results are shown in FIGS. 28A-F, 29A-F and 30A-F, where FIGS. 28A, 28D, 29A, 29D, 30A and 30D correspond to the grand average, FIGS. 28B, 28E, 29B, 29E, 30B and 30E correspond to the ERP of subject s1, and FIGS. 28C, 28F, 29C, 29F, 30C and 30F correspond to the ERP of subject sl. FIGS. 28A-F show fMRI slices, FIGS. 29A-C show the cortical potential maps for activation at a time point of 450 ms, FIGS. 29D-F show the cortical potential maps for activation at a time point of 700 ms, and FIGS. 30A-F show the cortical fMRI activation shown in FIGS. 28A-F superimposed on a three-dimensional synthetic brain model.

For the 450 ms time-point, strong parietal activation was observed for both the grand average and the single-subject ERPs. This is shown in the fMRI analysis and also in the BP-CPI estimated cortical maps. For the 700 ms time-point, more frontal activations were identified. High correlation was observed between the fMRI and ERPs, for both the grand average and the single-subject ERPs.

In this Example, the BP-CPI technique of the present embodiments was validated using simulative and experimental data. The simulative validation process showed a good match between the reference and the estimated cortical potentials. The estimated cortical potential maps can effectively reduce the “smearing” effect caused by the skull observed in the scalp potential maps, and recover the underlying brain electric activities with much improved spatial resolution. Simulations were performed with different source distributions in order to validate the technique of the present embodiments for important areas of the cortical surface.

The sensitivity analysis presented in this Example examined the competence of BP-CPI technique of the present embodiments to correctly estimate the cortical potentials while considering different real-life interferences and errors such as electrodes noise, displacement error, number of measurement sites and head model conductivity estimation errors. Measurement noise test showed that when dealing with SNR level of about 20 or above, very good estimation is found. This observation can optionally and preferably become an index rule when working with BP-CPI estimation of an ERP signals, which requires the SNR to be higher than 20. It was found by the Inventors that standard deviation of about 5 mm or less provides a sufficiently accurate estimation. It was found that configurations with 124 electrodes and 62 electrodes provide similar results, and are preferred over configurations with 29 or less electrodes. However, a number of electrodes which is less than 62 is also contemplated, optionally and preferably with a denser distribution compared to the standardized 10-20 system.

The BP-CPI of the present embodiments can be applied to EEG time signals acquired during a therapeutic procedure. In these embodiments, the BP-CPI can be used for estimating the effect of the therapeutic procedure procedures on the brain.

### Example 3

This Example provides a further study of the CPI technique of the present embodiments. The scalp potentials are back-projected onto the cortex surface using a realistic head model. The Laplace equation is solved by means of finite element method. A solution to the CPI problem is obtained by introducing of a cortical normal current estimation which is based on the same mechanism used in the surface Laplacian calculation with a scalp-cortex back-projection technique. In this Example, BP-CPI simulation results were validated versus an analytical solution of a spherical head model and against simulation results obtained using commercial electromagnetic simulations software (Sim4Life), on a realistic head model. The results show good cortical potentials estimation on spherical head model (CC=0.997), and realistic head model (CC>0.9) in the regions of interest (ROI).

Electrical brain activity is spatially distributed within the head structure and evolves with time. Nowadays, few modalities of functional brain imaging are available, including PET, SPECT, fMRI, MEG and EEG. Most of these modalities are very expensive, non-portable, ionizing or maintaining non-direct measurement of the brain electrical activity. Although these modalities provide sufficient spatial resolution, it was found by the Inventors that their temporal resolution of brain activity is insufficient, preventing researchers from capturing split-second seizure generation or cognitive bio-markers. Another advantage of the technique of the present embodiments is that it allows integration of monitoring and stimulation tools. This is because EEG is non-invasive, portable, passive and giving a direct measurement of the electrical potential signals on the scalp.

In this Example, the scalp potential distribution is back-projected to the cortex surface using an electroquasistatic (EQS) mechanism. Using the conductivity information of the realistic head model maintained from the single subject MRI T1 scan, along with scalp potentials measured using EEG electrodes, a cortical current estimation is introduced to generate a solution to the Laplace equation inside the volume above the cortical surface and below the scalp (the solution volume). It is known that if the Laplace equation is solved in a volume wrapped by known boundary conditions, the solution is single and unique [Yamashita Yasuo, 1982, IEEE Transactions on Biomedical Engineering, 11, pages 719-725].

The technique of the present embodiments makes use of the finite element method (FEM) to solve the Laplace equation in order to account for tissues with non-homogeneous conductivity properties. The BP-CPI estimates the cortical currents by employing the same mechanism used in the SL calculation with a back-projection technique to maintain an accurate estimation. The technique used in this Example requires a single iteration of the calculations.

### Materials and Methods

### The Back-Projection Solution

The volume conductor problem can be formulated in terms of a quasistatic Poisson equation. For a volume with no sources, the Poisson equation reduces to the Laplace equation which can be solved, with a unique solution, when the boundary condition on the volume boundaries are known [Yamashita 1982 supra]. It is assumed that the volume between the scalp and cortex surfaces, referred to in this example as the solution volume, is devoid of sources due to the fact that no neural cells lay in that region.

The Laplace equation formulation of this Example is the same as described in Example 1 above (see EQs. 1.1 and 1.2). In the backward problem solution, the Laplace equation is solved while the scalp potentials are known and the cortical boundary condition is estimated, as explained in Example 1 above (see EQs. 1.3 and 1.4).

In this Example, a physics based boundary condition is introduced on the cortical surface in order to give a full description of the back-projection problem and maintain a single and unique solution for the potentials in the entire solution volume. By solving the Laplace equation using the described scheme, the cortical potentials are captured due to both the normal and tangential potential derivatives. In order to maintain the scalp potentials p, EEG potentials are measured at electrodes sites and interpolated over all scalp surface mesh nodes using “thin plate” spline interpolation method [Soufflet et al., 1991, Electroencephalography and clinical Neurophysiology, 79, 5, pages 393-402] which is computationally fast.

Cortical Current Estimation

To estimate the cortical current the SL operator, calculated based on measured scalp potentials, is used, as follows:

$${σ}_{s} \underbrace{{∇}_{s}^{2} {Φ}_{s}}_{SL} = {∇}_{s} \cdot {σ}_{s} {∇}_{s} {Φ}_{s} = {∇}_{s} \cdot {J}_{s} \\ {I}_{in} = {\int }_{c} {J}_{n} dl = {\int }_{s} {∇}_{s} \cdot {J}_{s} dS = {σ}_{s} {\int }_{s} SLdS \qquad ( EQ . 3.1 )$$

where the scalp is divided into surface elements with area of ΔS, conductivity of σₛ, and potential of Φₛ. Iᵢₙ is the total normal current entering the element (with density Jₛ) and Jₙ is the tangential current exiting it. Thus, using these notations, Iᵢₙ=σₛ·SL·ΔS.

The SL operator estimates the normal currents flowing from the skull to the scalp layer by using the fact that any normal current reaching the scalp outer surface vanishes due to the boundary condition described in EQ. 1.2 and optionally and preferably transforms to the tangential direction. As in Example 1 above, the method described in [Le, 1994 supra] is used for calculating the Surface Laplacian. The method estimates the SL values through a local planar parametric space using Taylor expansion around each electrode site, with the least-squares technique. In addition, spatial low-pass filters were implemented pre and post-calculation, adapted to the head models optimized to cancel high frequency noises which can cause instability in the SL calculations. This procedure calculates the SL on the scalp surface. For the purpose of imposing cortical current as boundary condition, the Surface Laplacian results were projected onto the cortical surface, as described In Example 1, above.

Back-Projected SL Factorization

Once the surface Laplacian was calculated on the scalp surface and its projection on the cortical surface, the SL result optionally and preferably takes the form of current density including a projection factor (PF) with units of m²/Ω. The definition of the PF is PF=J_{cortex}/SL. It can be shown [Nunez 2006, supra] that:

J_{cortex}≈Jₖ≈Jₛ=(−σₛdₛ)SL (EQ. 3.2)

where Jₛ is the density of the current entering a scalp finite element cell, σₛ is its conductivity, dₛ is the local scalp thickness, and SL is the surface Laplacian operator calculated at the element center node. The current entering that element can be approximated by the normal current density Jₖ flowing from the skull into the scalp tissue. The cortex currents J_{cortex} can be considered as a good approximation to Jₖ while assuming high resistive skull layer. The PF can thus be defined as PF=−σₛdₛ.

Few general assumptions are made in the usage of the SL and PF: (i) head tissues are very thin relative to the electric field curvature, which is a reasonable assumption due to the very low frequency of the electric field and the dimensions of the head tissues and layer, about a few millimeters each; (ii) most of the current coming from within the brain is directed normal to both the cortical and skull surfaces, which is also a reasonable assumption due to low conductivity of the skull layer which reduces almost completely the spreading of currents, and the internal structure below the cortex surface which enables mostly normal currents to flow. As shown below, this assumption enables to back-project the SL onto the cortical surface, for both spherical and realistic head models.

Head Modeling and Electrodes Alignment

Automatic segmentation and meshing procedure was developed and included in the present study for the purpose of rapid single-subject realistic head model generation.

The procedure uses meshing and image processing developed tools, as well as the statistical parametric mapping (SPM) package [Ashburner et al., 2005, Neuroimage, 26, 3, pages 839-851], brain extraction tool (BET) software [Smith, 2002, Human brain mapping, 17, 3, pages 143-155], and iso2mesh toolbox [Q Fang and D Boas, “Tetrahedral mesh generation from volumetric binary and gray-scale images,” 2009 IEEE Int. Symposium on Biomedical Imaging, pp. 1142-1145, 2009]. The result was a tetrahedral head mesh, partitioned into different sub-divisions for various bone and tissues as depicted in FIGS. 6E-K. Each layer receives a homogeneous isotropic conductivity value. In this Example, the following conductivities were used 0.33, 0.0042, 0.0286 and 0.0042 [S/m], respectively for the scalp, the higher skull (compact bone), the intermediate skull (Diploe), and the lower skull (compact bone).

In addition, this head models take into account air cavities sparsely located within the head. The model in this example contained about 600 k elements, 10744 nodes on the scalp surface and 5910 nodes on the lower skull surface. The cerebral spinal fluid (CSF) and cortical surfaces were not included in the present example, but In some embodiments of the present invention at least one of the CSF and cortical surfaces is included. In the present Example, the estimation was projected only to the Lskull surface which is referred to herein as the cortical surface with cortical potentials. In is postulated that the potential distribution over the Lskull and over the cortex surface are not significantly different because of relatively high conductivity values in the volume below the Lskull surface.

The realistic head model generator used highly detailed T1 weighted MRI scans of a single subject with an in-plane resolution of 0.67 mm by 0.67 mm and slice thickness of 1 mm (3-D MP-RAGE, TR=2000 ms, TE=2.99 ms, 8° flip). The MRI scans were aligned and normalized according to the MNI template ICBM152, to allow co-registration and comparison between subjects.

A simplified three layer spherical head model was also generated as shown in FIG. 7A. The spherical head model has three spherical layers with radii of 8, 8.5 and 9 cm, and conductivity values of 1, 0.0125 and 1 S/m for the cortex, skull and scalp layers, respectively. Complete information of the realistic and spherical head models mesh is presented in Table 3, below where E, Nc, Nₛ, N and MEV represents total number of elements, number of cortex nodes, number of scalp nodes, total number of nodes and mean element volume in mm³, respectively.

TABLE 3

| Model | E | MEV | N_{c} | Nₛ | N |
|---|---|---|---|---|---|
| RHM | 499K | 4.38 | 3K | 6K | 82K |
| SPH | 300K | 2.9 | 2.5K | 2.5K | 53K |

The HydroCel Geodesic EEG sensor net (Electrical Geodesic Inc.) with 128 electrodes, denoted in this Example as EGI128 was used as the high resolution EEG system. The EGI128 has an inter-electrode spacing of about 2.5 cm. Even though it was suggested that for High-resolution EEG electrode spacing of 2 cm or lower is needed [Slutzky et al., 2010, Journal of neural engineering, 7, 2, page 026004], it likely provides an adequate representation of the scalp topography of most brain electrical events of interest to researchers and clinical practitioners.

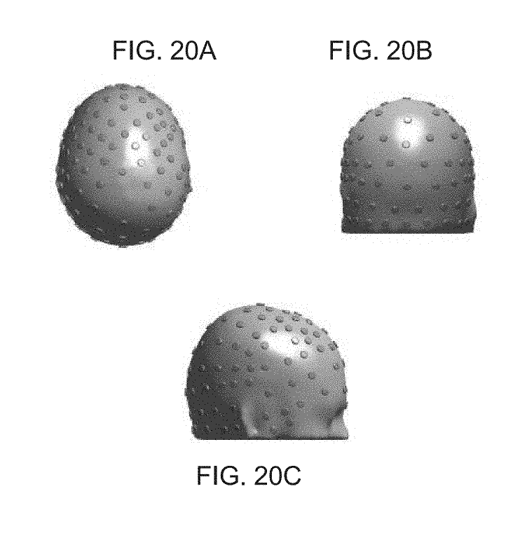

EEG electrodes location used in the head modeling and electrodes alignment procedure is originated in the general MNI coordinates given by the manufacturer. In order to align the system to the subject's scalp surface a simple optimization scheme that employed linear translation was applied so as to find the best fit between the subject's scalp to the electrodes locations. A single node on the scalp mesh was selected for each electrode by finding the closest scalp node to the radial projection of the electrode to the cortex surface. Results of the described alignment procedure are shown in FIGS. 20A-C.

Finite Element Method

The FEM was formulated as described in Example 1 above (see EQs. 1.5-1.18).

Validation by Forward Solution

In this Example, an accurate, stable and independent forward solution to the volume conductor problem in a realistic head was implemented. The solution was based on Sim4Life 1.2 software (by ZMT), a multiphysics simulation platform optimized for computational life sciences with strong support for simulations involving complex anatomical models such as head tissues. Its numerical algorithm is based on FEM.

The simulation performed with Sim4Life in this in this Example involved forward solution with the single purpose of generating a “true solution” to compare with the BP-CPI results. Different sources (electrical dipoles) distributions were placed inside the cortex volume, the sources strength and orientation was selected and the forward FEM solution was obtained with these sources excitations. Next, the scalp and cortical potentials were extracted from the solution. Scalp “true potentials” were used as the input to the BP-CPI algorithm and its output, the estimated cortical potentials, was compared to the cortex “true potentials”.

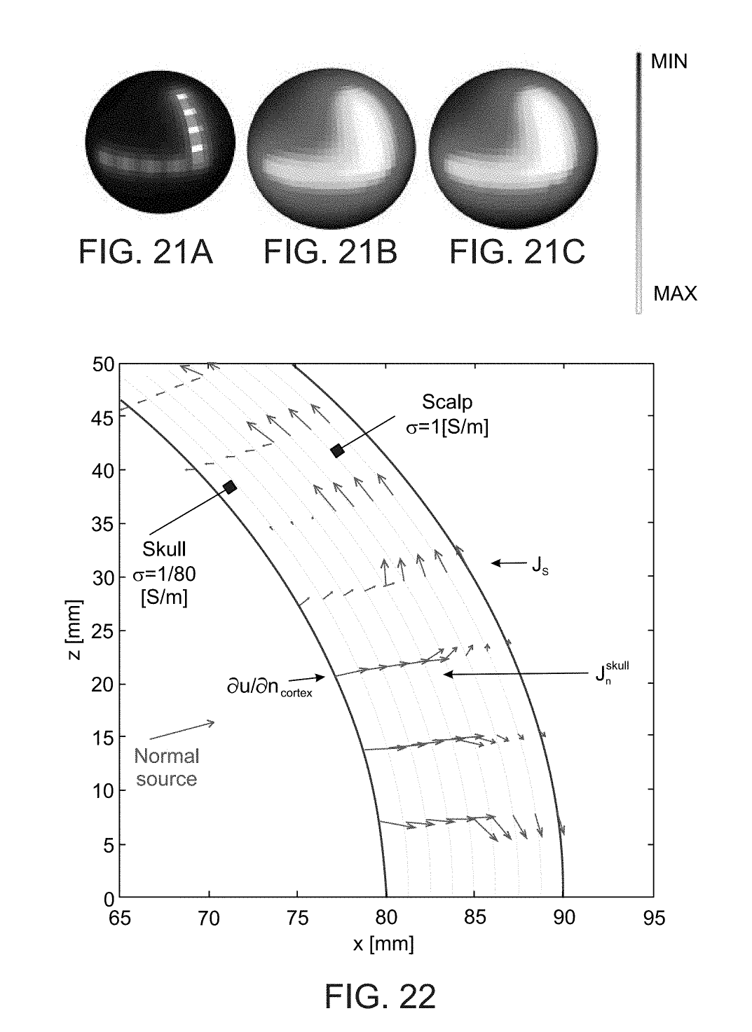

For the spherical head model, an analytical solution using harmonics spherical modes of the potential distribution for the case of a dipole placed in the cortex. The analytical potential distribution using spherical harmonics on the scalp and cortex surfaces was calculated for a nine, L shaped, normal dipole structure inside a spherical head model, then, the exact same source distribution was simulated using Sim4Life. Comparison results are shown in FIGS. 21A-C. Show are cortical potentials due to the L shape source distribution (FIG. 21A), and analytical (FIG. 21B) and Sim4Life (FIG. 21C) solutions on the scalp surface. As shown, the Sim4Life and the analytical solution are in good agreement.

The Pearson's correlation coefficient is used as a measure to quantify similarity. The CC definition is:

$$\begin{matrix}CC = \frac{{\sum }_{i = 1}^{N} ( {φ}_{i}^{A} - {\overline{φ}}^{A} ) ( {φ}_{i}^{B} - {\overline{φ}}^{B} )}{\sqrt{{\sum }_{i = 1}^{N} {( {φ}_{i}^{A} - {\overline{φ}}^{A} )}^{2}} \sqrt{{\sum }_{i = 1}^{N} {( {φ}_{i}^{B} - {\overline{φ}}^{B} )}^{2}}} & ( EQ . 3.3 ) \\ RE = \frac{1}{N} {\sum }_{i = 1}^{N} \vert \frac{{φ}_{i}^{A} - {φ}_{i}^{B}}{{φ}_{i}^{B}} \vert & ( EQ . 3.4 )\end{matrix}$$

where ϕᵢ^{A} and ϕᵢ^{B} are the potential value at the ith node of potential distribution A and B, respectively. The bar sign represents the mean of the vector, N is the total number of nodes on potential distributions and i runs over all nodes i=1 . . . N.

### Results

The correctness of the assumption that the normal cortical current estimation is correct was tested by comparing the “true” cortical currents to the estimated ones. This was done for the spherical head model, where an analytical solution exists, and for a realistic head model, where only numerical simulations are available for validation. Further investigation was done for two types of brain dipole sources oriented normally and tangentially to the cortex surface. A scheme of the test components is shown in FIG. 22. Shown is an XZ plane zoomed view of current distribution and components involved in the back-projection procedure. Lower and higher boundaries are the cortex and scalp surfaces. Normal dipole location and orientation is schematically drawn in red arrow.

The SL was performed over the smeared scalp potentials to estimate the skull normal currents Jₙ^{skull}, which are back-projected onto the cortex surface to find the BP-SL to be used as cortical normal potential derivative ∂u/∂n_{cortex}. The scheme shows the spreading of the scalp currents Jₛ when reaching the outer surface of the scalp. This fact is used in the SL calculation as depicted in EQs. 3.1 and 3.2.

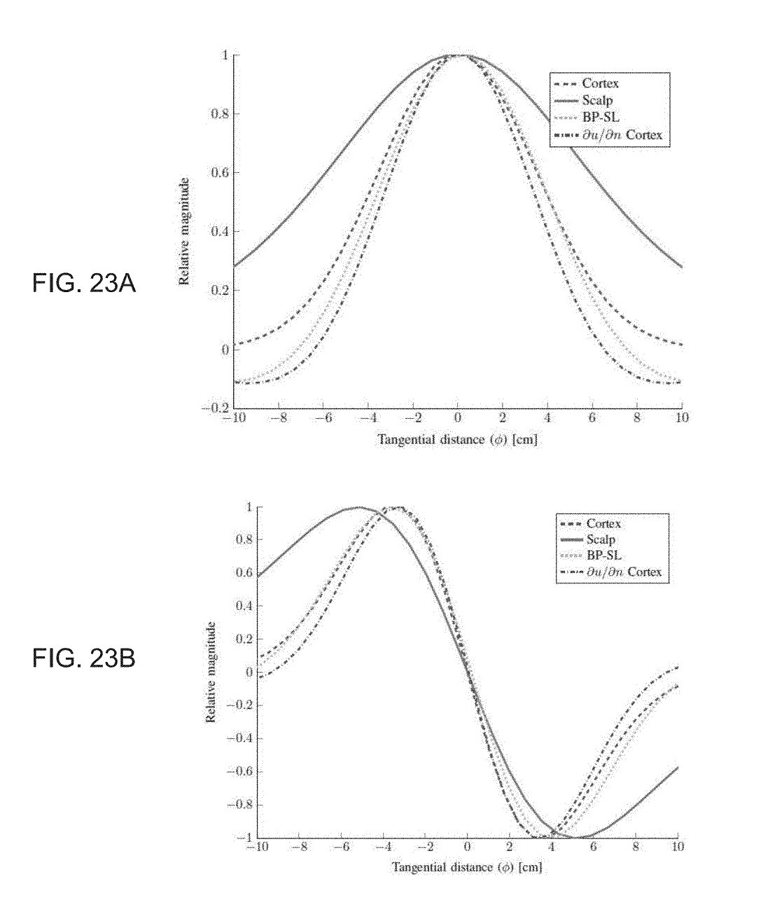

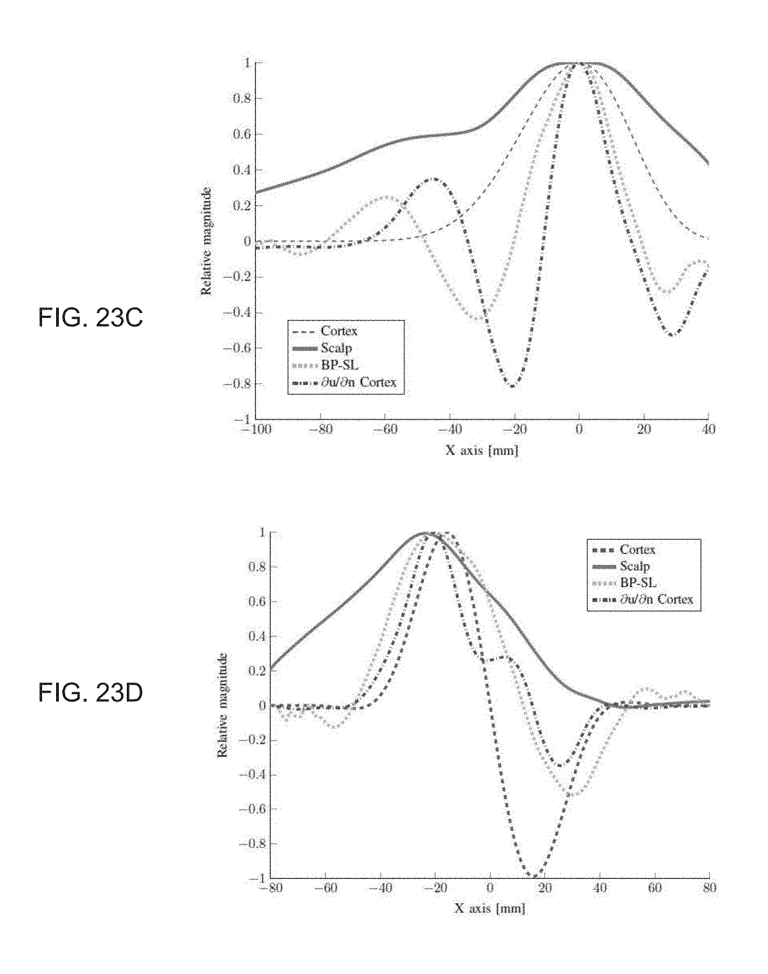

FIGS. 23A-D show results of validation of the Surface Laplacian as cortical estimator. Each variable was normalized with respect to its peak value. FIGS. 23A and 23B show normalized magnitude for each variable on spherical head model (with tangential distance axis), and FIGS. 23C and 23D show the same results on a realistic head model (with linear distance axis). All axis were aligned so the origin is above the source location, in the normal direction. The results show that the developed numerical SL, projected on the cortex surface, alongside with the analytical normalized normal oriented electric field (au/an) on cortex. As shown, a high correlation of the SL to the analytic solution is obtained for a spherical head model and a satisfying correlation for the realistic head model. The BP-SL diversion from the actual cortical currents can be related to non-perfect scalp-cortex projection that contains errors in some regions of the head. An example can be viewed in FIG. 23C where the right side of the BP-SL is a good estimation of the cortical currents, whereas the left regions (x<0), the BP-SL follows the cortical currents with about 15 mm offset between them.

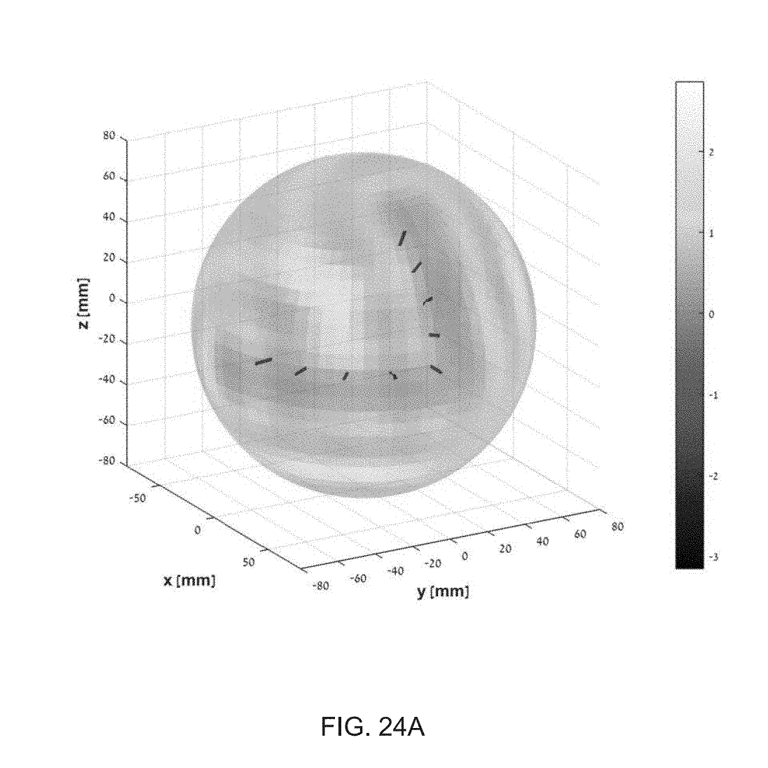

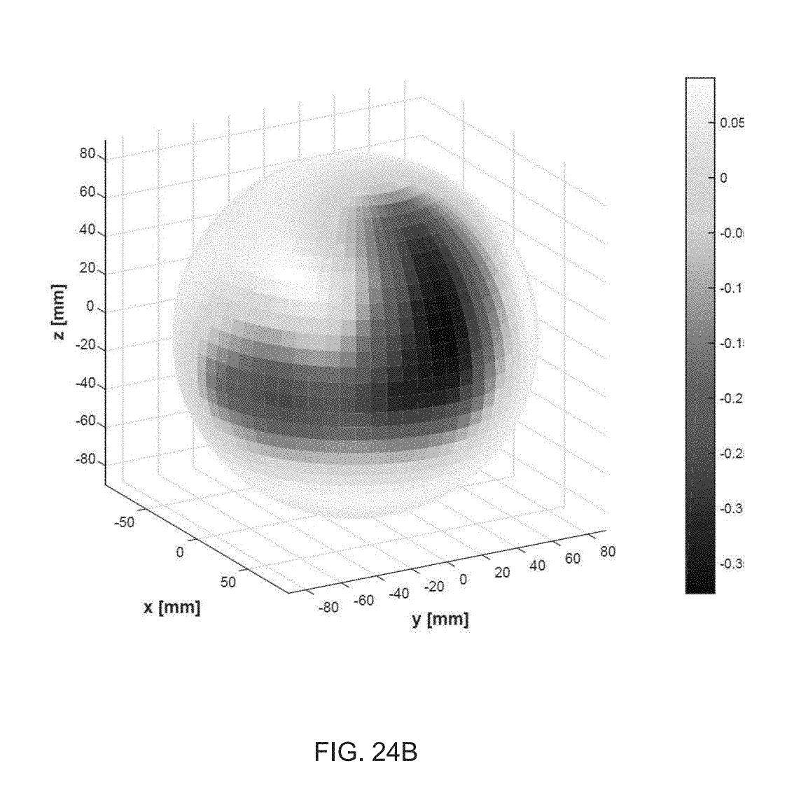

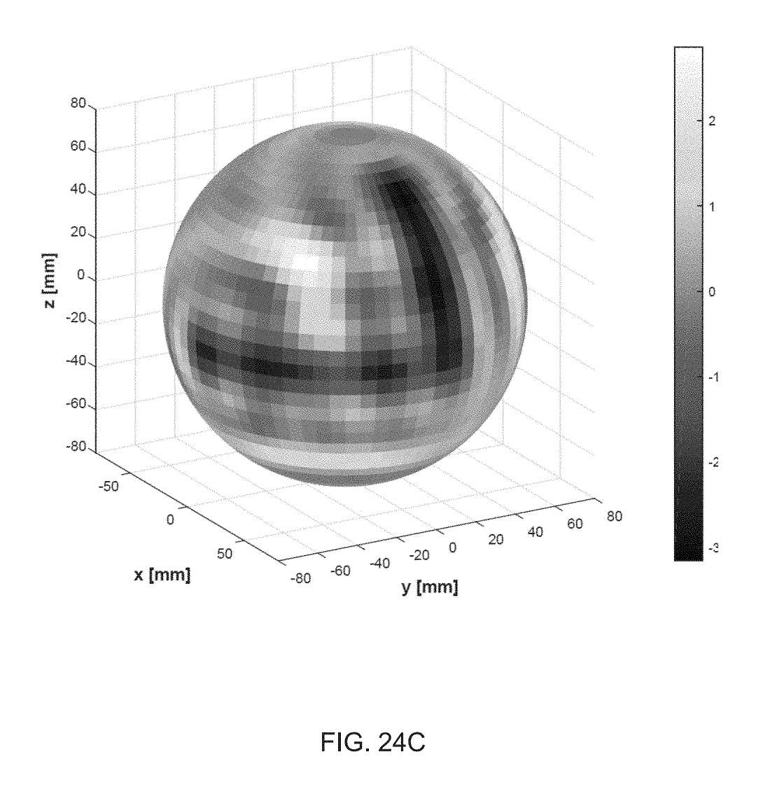

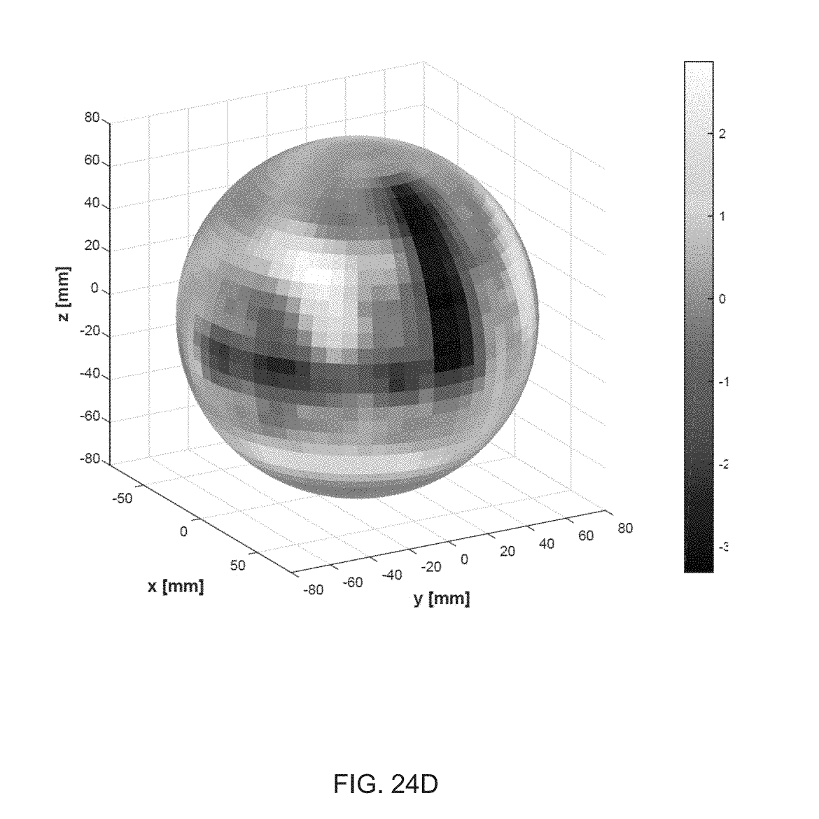

The source distribution (positive sources) is shown in FIG. 24A, similar to the one used in the validation of the forward solution described above, but with a source positioned 10 mm underneath the cortex surface instead of 5 mm as shown in FIG. 21A. The L shape sources distribution was selected as a deductive case for the BP-CPI on spherical head model. This is due to the proximity of the sources and their unique spatial position. In addition, it is straightforward to infer from these results to a single source or other source distributions. The analytical solutions of the scalp and cortex potentials distribution are shown in FIGS. 24B and 24C, respectively, and the estimated cortical potential distribution using BP-CPI is shown in FIG. 21D. As shown, the results are in a good agreement with the cortical potential resulting in a high CC between the analytical and estimated cortical potentials (CC=0.997) and are characterized by low RE (RE=0.002)

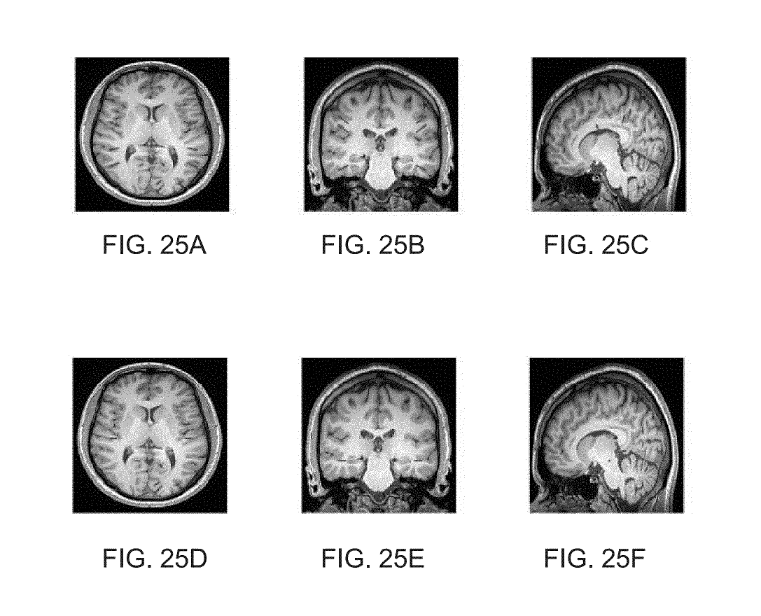

The accuracy of the BP-CPI technique of the present embodiments was studied for two typical simulated sources that are based on the brain activity excited by an auditory and visual stimuli, denoted in this example as auditory evoked potentials (AEP) and visually evoked potentials (VEP). Both sources distributions are shown in FIGS. 25A-F, incorporating seven VEP (FIGS. 25A-C) and two AEP (FIGS. 25D-F) source locations and orientations. FIGS. 25A and 25D show axial views, FIGS. 25B and 25E show coronal views, and FIGS. 25C and 25F show sagittal views of the subject MRI.

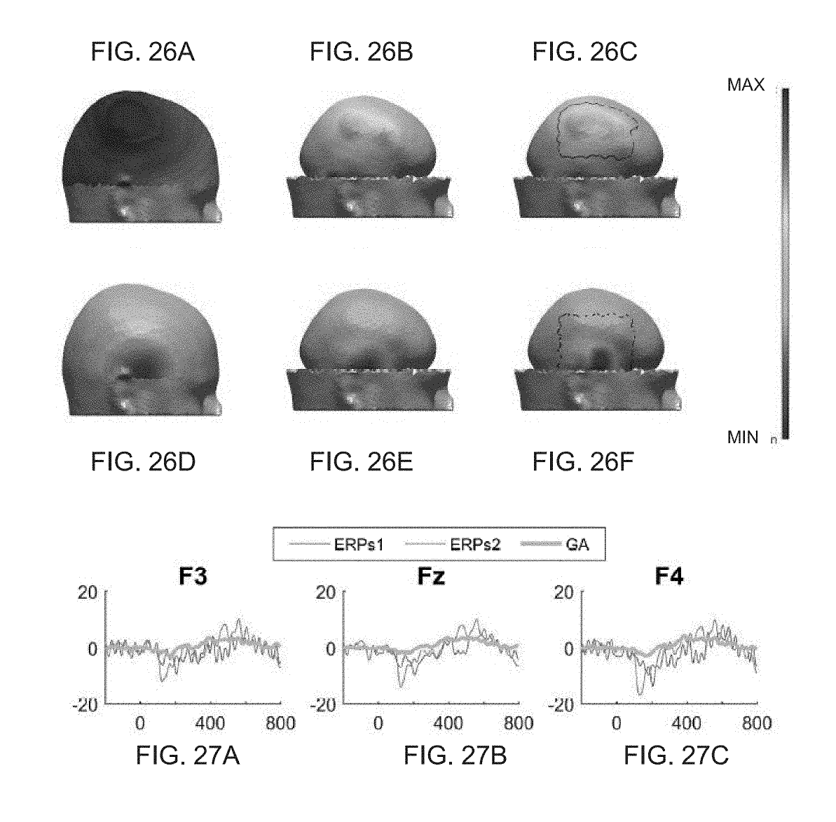

Forward solution was obtained for each of the selected VEP and AEP source distributions. The results are shown in FIGS. 26A-F, where FIG. 26A shows VEP signal on the scalp (forward solution), FIG. 26B shows reference VEP signal on the cortex, FIG. 26C shows VEP signal on cortex using BP-CPI, FIG. 26D shows AEP signal on the scalp (forward solution), FIG. 26E shows reference AEP signal on the cortex, and FIG. 26A shows AEP signal on cortex using BP-CPI. All scales are in [V] units. BP-CPI estimation and reference signals share the same scale. Scalp potentials were sampled at 124 sites and the BP-CPI algorithm was used to generate estimated cortical potentials.

In the case of VEP sources, it is shown that the two sources at the inferior fronto-parietal cortex were estimated with high similarity between the “true” cortical potentials and the BP-CPI estimation. The single source at the lateral inferior temporal cortex was localized, but with lower amplitude, due to the effect of high skull thickness and non-optimal SL projection factor in that region. In addition, the smearing effect generated by the skull which eliminates the cortical informative potentials is demonstrated when observing the scalp. The same inferences can be deduced while inspecting the results for the AEP. The BP-CPI localizes the cortical sources at the superior temporal lobe area with a good agreement to the “true” cortical potentials. Table 4, below, provides a quantitative measure for this agreement for both AEP and VEP cases. The CC was calculated for the whole cortical surface and for the ROI which contains the most energy in each case and is marked with a black dashed line in FIGS. 26C and 26F. The result of the BP-CPI thus provide accurate solution in regions where the signal is high, which are the regions of high clinical interest.

TABLE 4

| Model | CC | CC (ROI) |
|---|---|---|
| VEP | 0.78 | 0.94 |
| AEP | 0.5 | 0.95 |

This Example demonstrates that the BP-CPI technique of the present embodiments provides a fast (a few seconds on a laptop computer), non-parametric and accurate high resolution estimation for the cortical potentials on a realistic head model of the single subject.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### ADDITIONAL REFERENCES

[1] R. Van Uitert, C. Johnson, and L. Zhukov, “Influence of head tissue conductivity in forward and inverse magnetoencephalographic simulations using realistic head models”. IEEE Trans Biomed Eng. vol. 51, no. 12 pp. 2129-2137, 2004.
[2] M Thevenett, and J. Perniert, “The finite element method for a realistic head model of electrical brain activities: preliminary results”, Physiological measurements, vol. 12, Supp. A, pp. 89-94. 1991.
[3] V. Montes-Restrepo, “Influence of skull modeling approaches on EEG source localization”. Brain Topography, vol. 27, no. 1, pp. 95-111, 2014.

1. A method of estimating potential distribution over a cortical surface of a brain of a subject having a head, the method comprising:
obtaining encephalogram (EG) data recorded from a scalp surface of the head, and head model data describing a geometry of the head and electrical property distribution of tissues within the head;
calculating differentials of said EG data over the scalp surface, and projecting said differentials onto the cortical surface, based on said head model data;
calculating volumetric distribution of electrical potential between the cortex and scalp surfaces using said EG data and said projected differentials; and
estimating the potential distribution over the cortical surface based on said volumetric distribution.

2. The method according to claim 1, wherein said calculating said volumetric distribution comprises numerically solving a Laplace equation within a volume defined between said surfaces, under boundary conditions defined using said EG data and using said projected differentials.

3. The method according to claim 2, wherein one of said boundary conditions is a Dirichlet boundary condition at said and another one of said boundary conditions is a Neumann boundary condition.

4. The method according to claim 3, wherein said Dirichlet boundary condition is defined over said scalp surface, and said Neumann boundary condition is defined over said cortical surface.

5. The method according to claim 1, wherein said projecting comprises geometrical mapping of said differentials from the scalp surface onto the cortical surface, irrespectively of said electrical property distribution.

6. The method according to claim 1, wherein said projecting comprises geometrical mapping of said differentials from the scalp surface onto the cortical surface, and electrodynamic transformation of a value of said differentials based on said electrical property distribution.

7. The method according to claim 1, wherein said differentials comprise surface Laplacian.

8. The method according to claim 1, wherein said EG data comprises processed EG data that is interpolated over said scalp surface.

9. The method according to claim 1, further comprising processing said EG data to interpolate said EG data over said scalp surface.

10. The method according to claim 9, further comprising receiving input pertaining to a contact area of EG electrodes and correcting said interpolation based on said contact area.

11. The method according to claim 9, further comprising estimating displacement of EG electrodes and correcting said interpolation based on said estimated displacement.

12. The method according to claim 1, further comprising obtaining an image of the head, and constructing said head model from said image to provide said head model data.

13. The method according to claim 12, wherein said image comprises an MRI scan.

14. The method according to claim 12, wherein said constructing said head model comprises segmenting said image according to at least three tissue types, and assigning a predetermined value of said electrical property to each tissue type.

15. The method according to claim 1, wherein said head model data comprise data pertaining to air cavities located within said geometry of the head.

16. The method according to claim 15, wherein said air cavities are sparsely distributed within said geometry of the head.

17. The method according to claim 1, wherein said electrical property comprises at least one of electrical conductivity and electrical resistivity.

18. The method according to claim 12, further comprising displaying on a display device a graphical user interface (GUI) having a head model viewing region showing said head model, a user input control displaying changeable parameters characterizing said head model, and a calculation activation control, and repeating said construction of said head model using parameters in said user input control responsively to an activation of said control and to a change in said parameters.

19. The method according to claim 1, further comprising calculating a score describing said estimation.

20. The method according to claim 19, further comprising iteratively repeating said calculation of said volumetric distribution and estimation of the potential distribution over the cortical surface, until said score is within a predetermined score range.

21. The method according to claim 1, further comprising comparing said estimated potential distribution to a previously estimated potential distribution, and assessing a change in a condition of the subject based on said comparison.

22. The method according to claim 1, wherein said EG data is recorded during and/or after a treatment, and the method comprises comparing said estimated potential distribution to a previously estimated potential distribution, and assessing the effect of said treatment based on said comparison.

23. A system for estimating potential distribution over a cortical surface of a brain of a subject having a head, the system comprises a data processor configured for receiving encephalogram (EG) data recorded from a scalp surface of the head, and head model data describing a geometry of the head and electrical property distribution of tissues within the head, and executing the method according to claim 1.

24. A computer software product, comprising a computer-readable medium in which program instructions are stored, which instructions, when read by a data processor, cause the data processor to receive encephalogram (EG) data recorded from a scalp surface of the head, and head model data describing a geometry of the head and electrical property distribution of tissues within the head and to execute the method according to claim 1.
